(19) United States
(12) Patent Application Publication
(10) Pub. No.: US 2024/0336664 A1
(43) Pub. Date: Oct. 10, 2024

(54) SULFATED POLYPEPTIDES FOR SYSTEMIC DELIVERY

(71) Applicant: Adarsha Koirala, Framingham, MA (US)

(72) Inventors: Adarsha Koirala, Framingham, MA (US); Karen Ellen Petra De Ceunynck, Jamaica Plain, MA (US)

(21) Appl. No.: 18/294,862

(22) PCT Filed: Aug. 4, 2022
(86) PCT No.: PCT/US22/39413

§ 371 Date: Feb. 2, 2024

Related U.S. Application Data
(60) Provisional application No. 63/229,452, filed on Aug. 4, 2021.

(51) Int. Cl. C07K 14/47; A61K 9/00; A61K 38/00; A61K 48/00; C07K 1/107; C12N 15/87

(52) U.S. Cl. CPC C07K 14/4708; A61K 9/0019; A61K 48/005; C07K 1/1077; C12N 15/87; A61K 38/00; C07K 2319/09; C07K 2319/33; C07K 2319/35; C07K 2319/70; C07K 2319/80

(57) ABSTRACT

The present disclosure includes, among other things, methods and compositions for systemic (e.g., intravenous) delivery of sulfated polypeptide agents. In various embodiments, the present disclosure includes methods and compositions that reduce toxicity of systemic administration of polypeptide agents. In various embodiments, the present disclosure includes methods and compositions that cause or increase delivery of polypeptide agents to target cells, tissues, or organs (e.g., muscle, e.g., skeletal muscle, cardiac muscle, and/or diaphragm) following systemic (e.g., intravenous) delivery. In various embodiments, the present disclosure provides promoter sequences that achieve advantageous expression of operably linked coding sequences in muscle, e.g., in skeletal muscle, cardiac muscle, and/or diaphragm.

### CROSS REFERENCE TO RELATED APPLICATIONS

This application claims the benefit of U.S. Provisional Application No. 63/229,452 filed Aug. 4, 2021, the contents of which are hereby incorporated herein in their entirety.

### BACKGROUND

Polypeptide agents such as antibodies and replacement therapies can be useful in the treatment of a wide variety of conditions. Therapeutic efficacy of such polypeptide agents requires their delivery to therapeutically relevant cells and tissues.

### SUMMARY

The present disclosure provides, among other things, methods and compositions for delivery of polypeptide agents. In various embodiments, such methods and compositions include sulfated polypeptide agents. In various embodiments, methods and compositions of the present disclosure include systemic (e.g., intravenous) delivery of a sulfated polypeptide agent. As used to herein, a sulfated polypeptide agent refers to a polypeptide agent that includes at least one sulfated residue. The present disclosure further provides promoter nucleic acid sequences for expression of polypeptides in muscle.

The present disclosure includes the surprising discovery that systemic (e.g., intravenous) administration of a sulfated polypeptide agent can achieve delivery of the sulfated polypeptide agent to target cells, tissues, or organs (e.g., muscle, e.g., skeletal muscle, cardiac muscle, and/or diaphragm). In some embodiments, systemic (e.g., intravenous) delivery of a sulfated polypeptide agent can achieve delivery of a greater proportion of the agent to target cells, tissues, or organs (e.g., muscle, e.g., skeletal muscle, cardiac muscle, and/or diaphragm), as compared to the proportion delivered to the same cells or tissue(s) by a reference non-sulfated polypeptide, e.g., in or after reference period of time such as 1 hour, 2 hours, 3 hours, 6 hours, 1 day, 2 days, 3 days or 1 week.

The present disclosure includes the surprising discovery that systemic (e.g., intravenous) administration of a sulfated polypeptide agent can achieve reduced delivery of the sulfated polypeptide agent to liver and/or kidney. In some embodiments, systemic (e.g., intravenous) delivery of a sulfated polypeptide agent can achieve delivery of a reduced proportion of the agent to liver and/or kidney, as compared to the proportion delivered to the same cells, tissue(s), or organs by a reference non-sulfated polypeptide, e.g., in or after reference period of time such as 1 hour, 2 hours, 3 hours, 6 hours, 1 day, 2 days, 3 days or 1 week.

The present disclosure recognizes that reduced delivery to liver and/or kidney can reduce toxicity as compared to a reference. The present disclosure recognizes that reduced delivery to non-target tissues (including or excluding liver and/or kidney) can reduce toxicity as compared to a reference. The present disclosure recognizes that reduced delivery to non-target tissues (including or excluding liver and/or kidney) can increase therapeutic efficacy and/or reduce required dosages as compared to a reference. The present disclosure recognizes that increased delivery to target cells, tissues, or organs (e.g., muscle, e.g., skeletal muscle, cardiac muscle, and/or diaphragm) can increase therapeutic efficacy and/or reduce required dosages as compared to a reference. The present disclosure includes the unexpected discovery that such result is achieved by sulfation of polypeptide agents for systemic (e.g., intravenous) administration.

In at least one aspect, the present disclosure provides a method of delivering a polypeptide agent to one or more target cells, tissues, or organs of a subject, the method including systemically delivering a sulfated polypeptide agent.

In at least one aspect, the present disclosure provides a method of reducing toxicity of a polypeptide agent including systemically delivering a sulfated form of the polypeptide agent to a subject, where delivery of the sulfated polypeptide agent to, or accumulation of the sulfated polypeptide agent in, liver and/or kidney of the subject is reduced as compared to a reference non-sulfated form of the polypeptide agent.

In at least one aspect, the present disclosure provides a method of achieving delivery of a polypeptide agent to one or more target cells, tissues, or organs of a subject, the method including systemically delivering a sulfated form of the polypeptide agent to a subject, where delivery of the sulfated polypeptide agent to, or accumulation of the sulfated polypeptide agent in, the target cells, tissues, or organs of the subject is increased as compared to a reference non-sulfated form of the polypeptide agent.

In various embodiments, a sulfated polypeptide agent includes at least two, three, four, or five sulfated residues. In various embodiments, a sulfated polypeptide includes a sulfated tyrosine residue. In various embodiments, a sulfated polypeptide agent is an antibody or antibody fragment. In various embodiments, a sulfated polypeptide agent is an enzyme, structural protein, or viral vector. In various embodiments, a sulfated polypeptide agent is an engineered polypeptide including (i) a nucleic acid binding domain and (ii) at least one of a nucleic acid release domain, a nuclear localization signal, a stability domain, an oligomerization domain, and a targeting domain, optionally where the sulfated polypeptide is associated with one or more nucleic acid sequences. In various embodiments, a sulfated polypeptide agent is a mini-nucleosome core protein. In various embodiments, a sulfated polypeptide is a sulfated form of a polypeptide agent of which a non-sulfated form has been approved by the United States Food and Drug Administration (FDA) for administration to humans. In various embodiments, a sulfated polypeptide agent is a therapeutic agent. In various embodiments, a target cell, tissue, or organ is muscle tissue. In various embodiments, a target cell, tissue, or organ is skeletal muscle, cardiac muscle, diaphragm, or a combination thereof. In various embodiments, systemic delivery includes intravenous delivery. In various embodiments, a method of the present disclosure is characterized by increased delivery of the polypeptide agent to, or increased accumulation of the polypeptide agent in, the target cells, tissues, or organs as compared to a reference non-sulfated form of the polypeptide agent. In various embodiments, a method of the present disclosure is characterized by reduced delivery of the polypeptide agent to, or reduced accumulation of the polypeptide agent in, liver and/or kidney as compared to a reference non-sulfated form of the polypeptide agent.

In some embodiments, a sulfated polypeptide is associated with one or more nucleic acid sequences encoding one or more expression products and delivery results in expression of the expression products, optionally wherein expression of the expression products persists for at least 1 week, 2 weeks, 3 weeks, 1 month, 2 months, 3 months, 4 months, 5 months, 6 months, 7 months, 8 months, 10 months, 11 months, or 1 year after delivery.

In at least one aspect, the present disclosure provides a pharmaceutical composition including a sulfated polypeptide agent, where the composition is formulated for systemic delivery of the sulfated polypeptide to a subject. In various embodiments, a sulfated polypeptide agent includes at least two, three, four, or five sulfated residues. In various embodiments, a sulfated polypeptide includes a sulfated tyrosine residue. In various embodiments, a sulfated polypeptide agent is an antibody or antibody fragment. In various embodiments, a sulfated polypeptide agent is an enzyme, structural protein, or viral vector. In various embodiments, a sulfated polypeptide agent is an engineered polypeptide including (i) a nucleic acid binding domain and (ii) at least one of a nucleic acid release domain, a nuclear localization signal, a stability domain, an oligomerization domain, and a targeting domain, optionally where the sulfated polypeptide is associated with one or more nucleic acid sequences. In various embodiments, a sulfated polypeptide agent is a mini-nucleosome core protein. In various embodiments, a sulfated polypeptide is a sulfated form of a polypeptide agent of which a non-sulfated form has been approved by the United States Food and Drug Administration (FDA) for administration to humans. In various embodiments, a sulfated polypeptide agent is a therapeutic agent. In various embodiments, systemic delivery includes intravenous delivery. In various embodiments, a pharmaceutical composition of the present disclosure achieves increased delivery of the polypeptide agent to, or increased accumulation of the polypeptide agent in, the target cells, tissues, or organs as compared to a reference pharmaceutical composition including a non-sulfated form of the polypeptide agent. In various embodiments, a pharmaceutical composition of the present disclosure achieves reduced delivery of the polypeptide agent to, or reduced accumulation of the polypeptide agent in, liver and/or kidney as compared to a reference pharmaceutical composition including a non-sulfated form of the polypeptide agent.

In at least one aspect, the present disclosure provides a method of producing a nucleic acid vector for expression of an encoded polypeptide in muscle, the method comprising operably linking a promoter nucleic acid sequence having at least 80% sequence identity (e.g., 80%, 85%, 90%, 95%, 96%, 97%, 98%, 99%, or 100% sequence identity) with SEQ ID NO: 3 with a coding sequence.

In at least one aspect, the present disclosure provides a method of expressing a polypeptide in muscle, the method comprising administering to a cell, system, or subject a nucleic acid comprising a promoter nucleic acid sequence having at least 80% sequence identity (e.g., 80%, 85%, 90%, 95%, 96%, 97%, 98%, 99%, or 100% sequence identity) with SEQ ID NO: 3 operably linked with a coding sequence encoding the polypeptide.

In at least one aspect, the present disclosure provides a nucleic acid comprising a promoter nucleic acid sequence having at least 80% sequence identity (e.g., 80%, 85%, 90%, 95%, 96%, 97%, 98%, 99%, or 100% sequence identity) with SEQ ID NO: 3 operably linked with a coding sequence encoding a polypeptide for expression in muscle.

In various methods and compositions of the present disclosure, a sulfated polypeptide agent does not include (i) a nucleic acid binding domain and/or (ii) at least one of a nucleic acid release domain, a nuclear localization signal, a stability domain, an oligomerization domain, and a targeting domain. In various methods and compositions of the present disclosure, a sulfated polypeptide agent is not a mini-nucleosome core protein. In various embodiments of the present disclosure, the polypeptide agent is not a mini-nucleosome core protein and/or does not include a nucleic acid binding domain.

### Definitions

A, An, The: As used herein, “a”, “an”, and “the” refer to one or to more than one (i.e., to at least one) of the grammatical object of the article. By way of example, “an element” discloses embodiments of exactly one element and embodiments including more than one element.

About: As used herein, term “about”, when used in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referenced value.

Administration: As used herein, the term “administration” typically refers to administration of a composition to a subject or system (e.g., a plurality of cells, in vivo, in vitro, or ex vivo, e.g., where the plurality of cells includes one or more target and/or host cells) to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or include, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Agent: As used herein, the term “agent” may refer to any chemical entity, including without limitation any of one or more of an atom, molecule, compound, amino acid, polypeptide, nucleotide, nucleic acid, protein, protein complex, liquid, solution, saccharide, polysaccharide, lipid, or combination or complex thereof.

Amino acid: in its broadest sense, as used herein, refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with a typical or canonical amino acid structure. For example, in some embodiments, an amino acid can be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification can, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” can be used to refer to a free amino acid; in some embodiments it can be used to refer to an amino acid residue of a polypeptide.

Antibody: As used herein, the term “antibody” refers to a polypeptide that includes one or more canonical immunoglobulin sequence elements sufficient to confer specific binding to a particular antigen (e.g., a heavy chain variable domain, a light chain variable domain, and/or one or more CDRs). Thus, the term antibody includes, without limitation, human antibodies, non-human antibodies, synthetic and/or engineered antibodies, fragments thereof, and agents including the same. Antibodies can be naturally occurring immunoglobulins (e.g., generated by an organism reacting to an antigen). Synthetic, non-naturally occurring, or engineered antibodies can be produced by recombinant engineering, chemical synthesis, or other artificial systems or methodologies known to those of skill in the art.

As is well known in the art, typical human immunoglobulins are approximately 150 kD tetrameric agents that include two identical heavy (H) chain polypeptides (about 50 kD each) and two identical light (L) chain polypeptides (about 25 kD each) that associate with each other to form a structure commonly referred to as a “Y-shaped” structure. Typically, each heavy chain includes a heavy chain variable domain (VH) and a heavy chain constant domain (CH). The heavy chain constant domain includes three CH domains: CH1, CH2 and CH3. A short region, known as the “switch”, connects the heavy chain variable and constant regions. The “hinge” connects CH2 and CH3 domains to the rest of the immunoglobulin. Each light chain includes a light chain variable domain (VL) and a light chain constant domain (CL), separated from one another by another “switch.” Each variable domain contains three hypervariable loops known as “complement determining regions” (CDR1, CDR2, and CDR3) and four somewhat invariant “framework” regions (FR1, FR2, FR3, and FR4). In each VH and VL, the three CDRs and four FRs are arranged from amino-terminus to carboxy-terminus in the following order: FR1, CDR1, FR2, CDR2, FR3, CDR3, and FR4. The variable regions of a heavy and/or a light chain are typically understood to provide a binding moiety that can interact with an antigen. Constant domains can mediate binding of an antibody to various immune system cells (e.g., effector cells and/or cells that mediate cytotoxicity), receptors, and elements of the complement system. Heavy and light chains are linked to one another by a single disulfide bond, and two other disulfide bonds connect the heavy chain hinge regions to one another, so that the dimers are connected to one another and the tetramer is formed. When natural immunoglobulins fold, the FR regions form the beta sheets that provide the structural framework for the domains, and the CDR loop regions from both the heavy and light chains are brought together in three-dimensional space so that they create a single hypervariable antigen binding site located at the tip of the Y structure.

In some embodiments, an antibody is a polyclonal, monoclonal, monospecific, or multispecific antibody (e.g., a bispecific antibody). In some embodiments, an antibody includes at least one light chain monomer or dimer, at least one heavy chain monomer or dimer, at least one heavy chain-light chain dimer, or a tetramer that includes two heavy chain monomers and two light chain monomers. Moreover, the term “antibody” can include (unless otherwise stated or clear from context) any art-known constructs or formats utilizing antibody structural and/or functional features including without limitation intrabodies, domain antibodies, antibody mimetics, Zybodies®, Fab fragments, Fab′ fragments, F(ab′)2 fragments, Fd′ fragments, Fd fragments, isolated CDRs or sets thereof, single chain antibodies, single-chain Fvs (scFvs), disulfide-linked Fvs (sdFv), polypeptide-Fc fusions, single domain antibodies (e.g., shark single domain antibodies such as IgNAR or fragments thereof), cameloid antibodies, camelized antibodies, masked antibodies (e.g., Probodies®), affybodies, anti-idiotypic (anti-Id) antibodies (including, e.g., anti-anti-Id antibodies), Small Modular ImmunoPharmaceuticals (“SMIPs™”), single chain or Tandem diabodies (TandAb®), VHHs, Anticalins®, Nanobodies® minibodies, BiTE®s, ankyrin repeat proteins or DARPINs®, nanobody, Avimers®, DARTs, TCR-like antibodies, Adnectins®, Affilins®, Trans-Bodies®, Affibodies®, TrimerX®, MicroProteins, Fynomers®, Centyrins®, and KALBITOR®s, CARs, engineered TCRs, and antigen-binding fragments of any of the above.

In various embodiments, an antibody includes one or more structural elements recognized by those skilled in the art as a complementarity determining region (CDR) or variable domain. In some embodiments, an antibody can be a covalently modified (“conjugated”) antibody (e.g., an antibody that includes a polypeptide including one or more canonical immunoglobulin sequence elements sufficient to confer specific binding to a particular antigen, where the polypeptide is covalently linked with one or more of a therapeutic agent, a detectable moiety, another polypeptide, a glycan, or a polyethylene glycol molecule). In some embodiments, antibody sequence elements are humanized, primatized, chimeric, etc, as is known in the art.

An antibody including a heavy chain constant domain can be, without limitation, an antibody of any known class, including but not limited to, IgA, secretory IgA, IgG, IgE and IgM, based on heavy chain constant domain amino acid sequence (e.g., alpha (α), delta (δ), epsilon (ε), gamma (γ) and mu (μ)). IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. As used herein, a “light chain” can be of a distinct type, e.g., kappa (κ) or lambda (λ), based on the amino acid sequence of the light chain constant domain. In some embodiments, an antibody has constant region sequences that are characteristic of mouse, rabbit, primate, or human immunoglobulins. Naturally-produced immunoglobulins are glycosylated, typically on the CH2 domain. As is known in the art, affinity and/or other binding attributes of Fc regions for Fc receptors can be modulated through glycosylation or other modification. In some embodiments, an antibody may lack a covalent modification (e.g., attachment of a glycan) that it would have if produced naturally. In some embodiments, antibodies produced and/or utilized in accordance with the present invention include glycosylated Fc domains, including Fc domains with modified or engineered such glycosylation.

Antibodyfragment: As used herein, an “antibody fragment” refers to a portion of an antibody or antibody agent as described herein, and typically refers to a portion that includes an antigen-binding portion or variable region thereof Δn antibody fragment can be produced by any means. For example, in some embodiments, an antibody fragment can be enzymatically or chemically produced by fragmentation of an intact antibody or antibody agent. Alternatively, in some embodiments, an antibody fragment can be recombinantly produced (i.e., by expression of an engineered nucleic acid sequence. In some embodiments, an antibody fragment can be wholly or partially synthetically produced. In some embodiments, an antibody fragment (particularly an antigen-binding antibody fragment) can have a length of at least about 50, 60, 70, 80, 90, 100, 110, 120, 130, 140, 150, 160, 170, 180, 190 amino acids or more, in some embodiments at least about 200 amino acids.

Associated with: Two events or entities are “associated” with one another, as that term is used herein, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, or a combination thereof.

Between or From: As used herein, the term “between” refers to content that falls between indicated upper and lower, or first and second, boundaries (or “bounds”), inclusive of the boundaries. Similarly, the term “from”, when used in the context of a range of values, indicates that the range includes content that falls between indicated upper and lower, or first and second, boundaries, inclusive of the boundaries.

Bioavailability: As used herein, the term “bioavailability” can refer to the degree to which a substance, e.g., a polypeptide such as an antibody or antibody fragment, administered to an in vivo subject, becomes available to a tissue to which the substance is targeted (e.g., the bloodstream and/or plasma). Bioavailability can refer to the degree to which a substance that has been administered to an in vivo subject is delivered to blood of the subject. Bioavailability can refer to the ability of a substance to perform a function in the subject. Bioavailability can be measured in a number of ways, e.g., as the concentration of a substance in the bloodstream or plasma. In some embodiments, bioavailability can be assessed, for example, by comparing the “area under the curve” (AUC) in a plot of the plasma concentration as a function of time (area under the plasma concentration curve from time zero to a time where the plasma concentration returns to baseline levels). AUC can be calculated, for example, using the linear trapezoidal rule. “AUC0-t” refers to the area under the plasma concentration curve from time zero to a time, t, later, for example to the time of reaching baseline.

BiologicalSample: As used herein, the term “biological sample” typically refers to a sample obtained or derived from a biological source (e.g., a tissue or organism or cell culture) of interest, as described herein. In some embodiments, a biological source is or includes an organism, such as an animal or human. In some embodiments, a biological sample is or includes biological tissue or fluid. In some embodiments, a biological sample can be or include cells, tissue, or bodily fluid. In some embodiments, a biological sample can be or include blood, blood cells, cell-free DNA, free floating nucleic acids, ascites, biopsy samples, surgical specimens, cell-containing body fluids, sputum, saliva, feces, urine, cerebrospinal fluid, peritoneal fluid, pleural fluid, lymph, gynecological fluids, secretions, excretions, skin swabs, vaginal swabs, oral swabs, nasal swabs, washings or lavages such as a ductal lavages or broncheoalveolar lavages, aspirates, scrapings, or bone marrow. In some embodiments, a biological sample is or includes cells obtained from a single subject or from a plurality of subjects. A sample can be a “primary sample” obtained directly from a biological source, or can be a “processed sample” (e.g., a sample prepared from a primary sample, e.g. by a process such as isolation, e.g., of mRNA, DNA, or protein, by a process that modifies the primary sample's chemical structure, and/or by a process that produces a new or different composition that represents one or more components or properties of the primary sample). A biological sample can also be referred to as a “sample.”

Comparable: As used herein, the term “comparable” refers to members within sets of two or more conditions, circumstances, agents, entities, populations, etc., that may not be identical to one another but that are sufficiently similar to permit comparison there between, such that one of skill in the art will appreciate that conclusions can reasonably be drawn based on differences or similarities observed. In some embodiments, comparable sets of conditions, circumstances, agents, entities, populations, etc. are typically characterized by a plurality of substantially identical features and zero, one, or a plurality of differing features. Those of ordinary skill in the art will understand, in context, what degree of identity is required to render members of a set comparable. For example, those of ordinary skill in the art will appreciate that members of sets of conditions, circumstances, agents, entities, populations, etc., are comparable to one another when characterized by a sufficient number and type of substantially identical features to warrant a reasonable conclusion that differences observed can be attributed in whole or part to non-identical features thereof.

Corresponding to: As used herein, the term “corresponding to” may be used to designate the position/identity of a structural element in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of skill in the art appreciate that residues in a provided polypeptide or polynucleotide sequence are often designated (e.g., numbered or labeled) according to the scheme of a related reference sequence (even if, e.g., such designation does not reflect literal numbering of the provided sequence). By way of illustration, if a reference sequence includes a particular amino acid motif at positions 100-110, and a second related sequence includes the same motif at positions 110-120, the motif positions of the second related sequence can be said to “correspond to” positions 100-110 of the reference sequence. Those of skill in the art appreciate that corresponding positions can be readily identified, e.g., by alignment of sequences, and that such alignment is commonly accomplished by any of a variety of known tools, strategies, and/or algorithms, including without limitation software programs such as, for example, BLAST, CS-BLAST, CUDASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE. Two sequences can identified as corresponding if they are identical or if they share substantial identity, e.g., at least 80%, 85%, 90%, 91%, 92%, 93%, 94%, 95%, 96%, 97%, 98%, or 99% identity. In various embodiments, a nucleic acid sequence can correspond to a sequence that is identical or substantially identical (e.g., at least 80%, 85%, 90%, 91%, 92%, 93%, 94%, 95%, 96%, 97%, 98%, or 99% identical) to the complement of the nucleic acid sequence.

Engineered: As used herein, the term “engineered” refers to the aspect of having been manipulated by the hand of man. For example, a polynucleotide is considered to be “engineered” when two or more sequences, that are not linked together in that order in nature, are manipulated by the hand of man to be linked to one another in the engineered polynucleotide. Those of skill in the art will appreciate that an “engineered” nucleic acid or amino acid sequence can be a recombinant nucleic acid or amino acid sequence. In some embodiments, an engineered polynucleotide includes a coding sequence and/or a regulatory sequence that is found in nature operably linked with a first sequence but is not found in nature operably linked with a second sequence, which is in the engineered polynucleotide and operably linked in with the second sequence by the hand of man. In some embodiments, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution, deletion, or mating). As is common practice and is understood by those of skill in the art, progeny or copies, perfect or imperfect, of an engineered polynucleotide or cell are typically still referred to as “engineered” even though the direct manipulation was of a prior entity.

Identity: As used herein, the term “identity” refers to the overall relatedness between polymeric molecules, e.g., between nucleic acid molecules (e.g., DNA molecules and/or RNA molecules) and/or between polypeptide molecules. Methods for the calculation of a percent identity as between two provided sequences are known in the art. Calculation of the percent identity of two nucleic acid or polypeptide sequences, for example, can be performed by aligning the two sequences (or the complement of one or both sequences) for optimal comparison purposes (e.g., gaps can be introduced in one or both of a first and a second sequences for optimal alignment and non-identical sequences can be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, optionally taking into account the number of gaps, and the length of each gap, which may need to be introduced for optimal alignment of the two sequences. The comparison of sequences and determination of percent identity between two sequences can be accomplished using a computational algorithm, such as BLAST (basic local alignment search tool).

“Improve,” “increase,” “inhibit,” or “reduce”: As used herein, the terms “improve”, “increase”, “inhibit”, and “reduce”, and grammatical equivalents thereof, indicate qualitative or quantitative difference from a reference.

Operably linked: As used herein, “operably linked” refers to the association of at least a first element and a second element such that the component elements are in a relationship permitting them to function in their intended manner. For example, a nucleic acid regulatory sequence is “operably linked” to a nucleic acid coding sequence if the regulatory sequence and coding sequence are associated in a manner that permits control of expression of the coding sequence by the regulatory sequence. Those of skill in the art will appreciate that such control can be indirect, e.g., where a regulatory sequence recruits factors that impact expression of a coding sequence or where a regulatory sequence recruits factors or otherwise influences activity of a promoter that impacts or derives expression of a coding sequence. In some embodiments, an “operably linked” regulatory sequence is directly or indirectly covalently associated with a coding sequence (e.g., in a single nucleic acid). In some embodiments, a regulatory sequence controls expression of a coding sequence in trans and inclusion of the regulatory sequence in the same nucleic acid as the coding sequence is not a requirement of operable linkage.

Pharmaceutically acceptable: As used herein, the term “pharmaceutically acceptable,” as applied to one or more, or all, component(s) for formulation of a composition as disclosed herein, means that each component must be compatible with the other ingredients of the composition and not deleterious to the recipient thereof.

Pharmaceutically acceptable carrier: As used herein, the term “pharmaceutically acceptable carrier” refers to a pharmaceutically-acceptable material, composition, or vehicle, such as a liquid or solid filler, diluent, excipient, or solvent encapsulating material, that facilitates formulation of an agent (e.g., a pharmaceutical agent), modifies bioavailability of an agent, or facilitates transport of an agent from one organ or portion of a subject to another. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutical composition or formulation: As used herein, the term “pharmaceutical composition” or “formulation” refers to a composition in which a therapeutic agent is formulated together with one or more pharmaceutically acceptable carriers.

Polypeptide: As used herein, “polypeptide” refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may be or include of natural amino acids, non-natural amino acids, or both. In some embodiments, a polypeptide may be or include only natural amino acids or only non-natural amino acids. In some embodiments, a polypeptide can include D-amino acids, L-amino acids, or both. In some embodiments, a polypeptide may include only L-amino acids. In some embodiments, a polypeptide may include one or more pendant groups or other modifications, e.g., one or more amino acid side chains, e.g., at the polypeptide's N-terminus, at the polypeptide's C-terminus, at non-terminal amino acids, or at any combination thereof. In some embodiments, such pendant groups or modifications may be selected from acetylation, amidation, lipidation, methylation, phosphorylation, glycosylation, glycation, sulfation, mannosylation, nitrosylation, acylation, palmitoylation, prenylation, pegylation, etc., including combinations thereof. In some embodiments, a polypeptide may be cyclic, and/or may include a cyclic portion.

In some embodiments, the term “polypeptide” may be appended to a name of a reference polypeptide, activity, or structure to indicate a class of polypeptides that share a relevant activity or structure. For such classes, the present specification provides and/or those skilled in the art will be aware of exemplary polypeptides within the class whose amino acid sequences and/or functions are known. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class. For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that can in some embodiments be or include a characteristic sequence element) that shows very high sequence identity, often greater than 90% or even 95%, 96%, 97%, 98%, or 99%. Such a conserved region usually encompasses at least 3-4 and in some instances up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide can be or include a fragment of a parent polypeptide. In some embodiments, a useful polypeptide may be or include a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Promoter: As used herein, a “promoter” or “promoter sequence” can be a DNA regulatory region that directly or indirectly (e.g., through promoter-bound proteins or substances) participates in initiation and/or processivity of transcription of a coding sequence. A promoter may, under suitable conditions, initiate transcription of a coding sequence upon binding of one or more transcription factors and/or regulatory moieties with the promoter. A promoter that participates in initiation of transcription of a coding sequence can be “operably linked” to the coding sequence. In certain instances, a promoter can be or include a DNA regulatory region that extends from a transcription initiation site (at its 3′ terminus) to an upstream (5′ direction) position such that the sequence so designated includes one or both of a minimum number of bases or elements necessary to initiate a transcription event. A promoter may be, include, or be operably associated with or operably linked with, expression control sequences such as enhancer and repressor sequences. In some embodiments, a promoter may be inducible. In some embodiments, a promoter may be a constitutive promoter. In some embodiments, a conditional (e.g., inducible) promoter may be unidirectional or bi-directional. A promoter may be or include a sequence identical to a sequence known to occur in the genome of particular species. In various embodiments a promoter is identified as a sequence including a given number of nucleotides that are immediately upstream of a transcription start site of a coding sequence of a known nucleic acid sequence (e.g., a genomic sequence), e.g., where the number of nucleotides can be 100 bp, 200 bp, 500 bp, 1 kb, 2 kb, 3 kb, 4 kb, or 5 kb. In some embodiments, a promoter can be or include a hybrid promoter, in which a sequence containing a transcriptional regulatory region can be obtained from one source and a sequence containing a transcription initiation region can be obtained from a second source. Systems for linking control elements to coding sequence within a transgene are well known in the art (general molecular biological and recombinant DNA techniques are described in Sambrook, Fritsch, and Maniatis, Molecular Cloning: A Laboratory Manual, Second Edition, Cold Spring Harbor Laboratory Press, Cold Spring Harbor, N Y, 1989). Those of skill in the art are familiar with a wide variety of known promoters and various uses and/or characteristics thereof.

Reference: As used herein, “reference” refers to a standard or control relative to which a comparison is performed. For example, in some embodiments, an agent, sample, sequence, subject, animal, or individual, or population thereof, or a measure or characteristic representative thereof, is compared with a reference, an agent, sample, sequence, subject, animal, or individual, or population thereof, or a measure or characteristic representative thereof. In some embodiments, a reference is a measured value. In some embodiments, a reference is an established standard or expected value. In some embodiments, a reference is a historical reference. A reference can be quantitative of qualitative. Typically, as would be understood by those of skill in the art, a reference and the value to which it is compared represents measure under comparable conditions. Those of skill in the art will appreciate when sufficient similarities are present to justify reliance on and/or comparison. In some embodiments, an appropriate reference may be an agent, sample, sequence, subject, animal, or individual, or population thereof, under conditions those of skill in the art will recognize as comparable, e.g., for the purpose of assessing one or more particular variables (e.g., presence or absence of an agent or condition), or a measure or characteristic representative thereof.

Sample: As used herein, the term “sample” typically refers to an aliquot of material obtained or derived from a source of interest. In some embodiments, a source of interest is a biological or environmental source. In some embodiments, a sample is a “primary sample” obtained directly from a source of interest. In some embodiments, as will be clear from context, the term “sample” refers to a preparation that is obtained by processing of a primary sample (e.g., by removing one or more components of and/or by adding one or more agents to a primary sample). Such a “processed sample” can include, for example cells, nucleic acids, or proteins extracted from a sample or obtained by subjecting a primary sample to techniques such as amplification or reverse transcription of nucleic acids, isolation and/or purification of certain components, etc.

Subject: As used herein, the term “subject” refers to an organism, typically a mammal (e.g., a human, rat, or mouse). In some embodiments, a subject is suffering from a disease, disorder or condition. In some embodiments, a subject is susceptible to a disease, disorder, or condition. In some embodiments, a subject displays one or more symptoms or characteristics of a disease, disorder or condition. In some embodiments, a subject is not suffering from a disease, disorder or condition. In some embodiments, a subject does not display any symptom or characteristic of a disease, disorder, or condition. In some embodiments, a subject has one or more features characteristic of susceptibility to or risk of a disease, disorder, or condition. In some embodiments, a subject is a subject that has been tested for a disease, disorder, or condition, and/or to whom therapy has been administered. In some instances, a human subject can be interchangeably referred to as a “patient” or “individual.” A subject administered an agent associated with treatment of a disease, disorder, or condition with which the subject is associated can be referred to as a subject in need of the agent, i.e., as a subject in need thereof.

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Therapeutically effective amount: As used herein, “therapeutically effective amount” refers to an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that a therapeutically effective amount does not necessarily achieve successful treatment in every particular treated individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Treatment: As used herein, the term “treatment” (also “treat” or “treating”) refers to administration of a therapy that partially or completely alleviates, ameliorates, relieves, inhibits, delays onset of, reduces severity of, and/or reduces incidence of one or more symptoms, features, and/or causes of a particular disease, disorder, or condition, or is administered for the purpose of achieving any such result. In some embodiments, such treatment can be of a subject who does not exhibit signs of the relevant disease, disorder, or condition and/or of a subject who exhibits only early signs of the disease, disorder, or condition. Alternatively or additionally, such treatment can be of a subject who exhibits one or more established signs of the relevant disease, disorder and/or condition. In some embodiments, treatment can be of a subject who has been diagnosed as suffering from the relevant disease, disorder, and/or condition. In some embodiments, treatment can be of a subject known to have one or more susceptibility factors that are statistically correlated with increased risk of development of the relevant disease, disorder, or condition.

### BRIEF DESCRIPTION OF THE DRAWING

FIG. 1 is a schematic showing sulfated and non-sulfated forms of a polypeptide agent, where the sulfated polypeptide agent includes a sulfated tyrosine (Y) residue.

FIG. 2 is a graph showing luciferase expression from intravenous (IV) administration of sulfated and non-sulfated forms of a polypeptide agent in left gastrocnemius muscle, where the polypeptide agent is a vector for delivery of associated nucleic acids and where the polypeptide agents were associated with nucleic acids encoding luciferase.

FIG. 3 is a graph showing luciferase expression from intravenous (IV) administration of sulfated and non-sulfated forms of a polypeptide agent in heart, where the polypeptide agent is a vector for delivery of associated nucleic acids and where the polypeptide agents were associated with nucleic acids encoding luciferase.

FIG. 4 is a blot showing expression of full length human dystrophin following intramuscular injection of a polypeptide agent associated with nucleic acid molecules encoding human dystrophin (DMD).

FIG. 5 is a chart showing CK levels in serum by absorbance at 450, where CK levels are positively correlated with disease severity.

FIG. 6 is a chart showing expression from certain muscle promoters in C2C12 mouse myoblasts.

FIG. 7 is pair of charts showing relative light units (RLU) in muscle tissues (tibialis anterior and gastrocnemius, respectively) following administration of a nucleic acid encoding luciferase associated with sulfated 118 polypeptide or unsulfated control polypeptide.

FIG. 8 is a chart showing levels of creatine kinase (CK) measured after a single dose or triple dose of intravenous injection of a nucleic acid encoding dystrophin associated with sulfated 118 polypeptide, as compared to sham-treated controls.

FIG. 9 is a pair of charts showing levels of creatine kinase (CK) measured 1 month post injection and 7.5 months post injection, respectively, of a nucleic acid encoding dystrophin associated with sulfated 118 polypeptide, at a low dose or a high dose,

FIG. 10 is blot showing expression of dystrophin in mice intravenously injected with a nucleic acid encoding dystrophin associated with sulfated 118 polypeptide.

### DETAILED DESCRIPTION

The present disclosure includes, among other things, methods and compositions for systemic delivery of polypeptide agents. In various embodiments, the present disclosure includes methods and compositions that reduce toxicity of systemic administration of polypeptide agents. In various embodiments, the present disclosure includes methods and compositions that cause or increase delivery of polypeptide agents to target cells, tissues, or organs (e.g., muscle, e.g., skeletal muscle, cardiac muscle, and/or diaphragm) following systemic (e.g., intravenous) delivery. In various embodiments, the present disclosure provides promoter sequences that achieve advantageous expression of operably linked coding sequences in muscle, e.g., in skeletal muscle, cardiac muscle, and/or diaphragm.

Without wishing to be bound by any particular scientific theory, the present disclosure provides, among other things, that sulfated polypeptide agents (e.g., mini-nucleosome core proteins) that are associated with nucleic acids can compact nucleic acids and delivery, or increase delivery of associated nucleic acids to target cells, tissues, or organs (e.g., muscle, e.g., skeletal muscle, cardiac muscle, and/or diaphragm) following systemic (e.g., intravenous) delivery.

Without wishing to be bound by any particular scientific theory, various advantages properties associated with sulfated polypeptide agents can result in whole or part from reduced accumulation in the liver, e.g., by increased escape from the liver, as compared to a non-sulfated reference. Such advantageous properties can include, for example, increased tropism for target cells, tissues, or organs (e.g., muscle, e.g., skeletal muscle, cardiac muscle, and/or diaphragm). Without wishing to be bound by any particular scientific theory, the present disclosure provides, sulfation of polypeptide agents can enhance, as one example, muscle tropism of polypeptide agents. Such enhanced muscle tropism can be advantageous, e.g., where a sulfated polypeptide agent is systemically (e.g., intravenously) administered to a subject.

Exemplary polypeptide agents of the present disclosure, any of which can be sulfated in accordance with the present disclosure and therefore be sulfated polypeptide agents, include without limitation antibodies, antibody fragments, enzymes, structural proteins, and mini-nucleosome core proteins. In some embodiments, the present disclosure includes sulfation of lipid nanoparticles or polypeptides associated therewith (e.g., lipid nanoparticles modified with peptides that are or can be sulfated polypeptides, such as sulfated targeting polypeptides), In some embodiments, polypeptide agents of the present disclosure, any of which can be sulfated in accordance with the present disclosure and therefore be sulfated polypeptide agents, include without limitation polypeptide agents approved by the United States Food and Drug Administration (FDA) for administration to humans (e.g., in a non-sulfated form). In various embodiments, a sulfated polypeptide agent is a therapeutic agent. In some embodiments, a sulfated polypeptide agent is a replacement therapy (e.g., an enzyme replacement therapy) or augmentation therapy. In various embodiments, sulfated polypeptide agents provided herein can be applied to rescue deleterious effects of genetic mutations that cause diseases including, without limitation, Cystic fibrosis, Duchenne muscular dystrophy, Stargardt's disease, Age-related macular degeneration, Huntington, Hemophilia A, Spinal muscular atrophy, Usher syndrome etc. In such diseases, a genetic mutation renders a gene nonfunctional or not available. In such cases, providing polypeptides encoded by corresponding non-mutant or healthy forms of the mutated gene may be beneficial to subjects.

Exemplary polypeptide agents of the present disclosure further include viral vectors. Exemplary viral vectors include without limitation adeno-associated vectors (AAVs), lentiviral vectors.

Sulfation refers to the covalent linkage of sulfate to an amino acid residue, typically a tyrosine (Y) residue. Sulfation or sulfurylation or sulfonation includes enzyme catalyzed conjugation of a sulfo group to another biomolecule. Tyrosine sulfation is the most common posttranslational modification observed in tyrosines. Tyrosines can be artificially sulfated, e.g., for use in medicine and other purposes. In certain embodiments, an amino acid immediately N-terminal to a sulfated tyrosine in a polypeptide is an amino acid selected from E, N, S, H, V, and D, and/or an amino acid immediately C-terminal to a sulfated tyrosine in a polypeptide is an amino acid selected from E, L, D, Q, P, T, R and Y.

Methods and compositions of the present disclosure can be used to treat various conditions that can benefit from delivery of a polypeptide agent, e.g., a sulfated form a polypeptide agent that is a therapeutic agent. To provide a non-limiting example, many muscular dystrophies can be treated by gene therapy. Gene therapy works by delivering nucleic acids that encode and/or express therapeutic nucleic acid products to tissues in subjects in need thereof. Factors impacting gene delivery via systemic administration include accumulation of vectors in the liver and insufficient targeting to muscle cells. Strategies to escape the liver would enhance the efficacy and safety of all gene therapies. Sulfated polypeptide agents of the present disclosure can include therapeutic polypeptides useful to treat muscular dystrophies direct, or can be vectors for delivery of nucleic acids encoding therapeutic polypeptides, the sulfation of which vectors provides advantages for delivery of associated nucleic acids.

It will be appreciated from the present disclosure that methods and compositions provided herein can be useful to treat a variety of conditions for which delivery of polypeptide (either directly as a sulfated therapeutic polypeptide agent or indirectly by use of a sulfated vector that delivers a nucleic acid encoding a therapeutic polypeptide agent) can be therapeutic. Examples of such conditions include, without limitation, (i) Becker Muscular Dystrophy (for which, to the present inventor's knowledge, there is currently no sufficiently effect treatment), (ii) Limb-Girdle muscular dystrophy (for which, to the present inventor's knowledge, there is currently no sufficiently effect treatment; estimated at lin every 14,500 to 1 in 123,000; cardiac issues), (iii) Myotonic Dystrophy Type 1 (Steinert's disease; 1/8000 rare, neuromuscular disease that affects multiple organ systems (eye, heart, endocrine system, CNS), and is characterized primarily by myotonia and progressive muscle wasting and weakness), (iv) SMA (Spinal muscular atrophy; treatment available but very expensive) (v) Pompe disease (rare; estimated at 1 in every 40,000 births; disables the heart and skeletal muscles; It is caused by mutations in a gene that makes an enzyme called acid alpha-glucosidase (GAA); Cardiac issues are common); (vi) X-linked myotubular myopathy; and (vii) Cardiomyopathies.

In certain embodiments, a sulfated polypeptide agent is a mini-nucleosome core protein. Mini-nucleosome core proteins are described, for example, in PCT Publication WO 2020/097235, entitled Mini-Nucleosome Core Proteins and use in Nucleic Acid Delivery, filed as PCT/US19/60119 on Nov. 6, 2019, which is incorporated herein by reference in its entirety and particularly with respect to mini-nucleosome core proteins and sequences thereof. In certain embodiments, a sulfated polypeptide agent of the present disclosure can be a modified mini-nucleosome core protein. Modified mini-nucleosome core proteins are described, for example, in International Application No. PCT/US21/26917, entitled Modified Mini-Nucleosome Core Proteins and use in Nucleic Acid Delivery, filed Apr. 12, 2021, which is incorporated herein by reference in its entirety and particularly with respect to modified mini-nucleosome core proteins and sequences thereof.

While mini-nucleosome core proteins are disclosed in WO 2020/097235, which is incorporated herein by reference in its entirety and particularly with respect to mini-nucleosome core proteins and sequences thereof, a partial discussion of mini-nucleosome core proteins is provided below by way of non-limiting exemplification.

In various embodiments, a mini-nucleosome core protein is an engineered polypeptide including (i) a nucleic acid binding domain and (ii) at least one of a nucleic acid release domain, a nuclear localization signal, a stability domain, an oligomerization domain, and a targeting domain, e.g., as disclosed in WO 2020/097235. In various such embodiments, the nucleic acid binding domain is a nucleic acid binding domain derived from a histone polypeptide sequence. In various such embodiments, the nucleic acid binding domain is or includes the amino acid sequence KRHRK (SEQ ID NO: 11). In various such embodiments, the nucleic acid binding domain is or includes an amino acid sequence that includes KRHRK (SEQ ID NO: 11), RRRRR (SEQ ID NO: 12), RRLARR (SEQ ID NO: 13), KKAKAAAKPKK (SEQ ID NO: 14), KKDGKKRKR (SEQ ID NO: 15), KKKLK (SEQ ID NO: 16), KKRIRK (SEQ ID NO: 17), RKKSK (SEQ ID NO: 18), KKPKK (SEQ ID NO: 19), or a combination thereof.

In various embodiments, the engineered polypeptide includes a targeting domain. In various such embodiments, the targeting domain is a cell attachment targeting domain, a beta galactose binding domain, a fucose binding domain, a heparin binding domain, a sialic acid binding domain, a glycoprotein binding domain, a carbohydrate binding domain, a lysophosphatidic acid binding domain, a cAMP binding domain, a hyaluronan binding domain, a chondroitin sulfate binding domain, an integrin binding domain, a nucleolin binding domain, a collagen binding domain, a clathrin binding domain, a Fc receptor binding domain, an actin binding domain, an endocytosis motif, a nuclear localization signal, or a combination thereof. In various embodiments, the cell attachment targeting domain is or includes an amino acid sequence that includes WGREERQ (SEQ ID NO: 20), NTQIH (SEQ ID NO: 21), WNNKTPH (SEQ ID NO: 22), TPH (SEQ ID NO: 23), VNRWS (SEQ ID NO: 24), XBBBXXBX (SEQ ID NO: 25), ARKKAAKA (SEQ ID NO: 26), QRR (SEQ ID NO: 27), SRR (SEQ ID NO: 28), WEPSRPFPVD (SEQ ID NO: 29), HRRTRKAPKRIRLPHIR (SEQ ID NO: 30), KRTGQYKLGSKTGPGQK (SEQ ID NO: 31), KKTK (SEQ ID NO: 32), KLRSQLVKK (SEQ ID NO: 33), RRRCGQKKK (SEQ ID NO: 34), BX(7)B (SEQ ID NO: 35), RIQNLLKITNLRIKFVK (SEQ ID NO: 36), KKEKDIMKKTI (SEQ ID NO: 37), KGE (SEQ ID NO: 38), RGD (SEQ ID NO: 39), RGDS (SEQ ID NO: 40), TTVVNPKYEGK (SEQ ID NO: 41), ERMSQIKRLLS (SEQ ID NO: 42), WRHRARS (SEQ ID NO: 43), GFOGER (SEQ ID NO: 44), LFDLM (SEQ ID NO: 45), WGREERQ (SEQ ID NO: 46), QSTEKRG (SEQ ID NO: 47), LPNTG (SEQ ID NO: 48), or a combination thereof.

In various embodiments, the targeting domain is an internalization domain. In various embodiments, the internalization domain is or includes an amino acid sequence that includes FXDXF (SEQ ID NO: 49), PPSY (SEQ ID NO: 50), FEDNFVP (SEQ ID NO: 51), YIRV (SEQ ID NO: 52), YADW (SEQ ID NO: 53), YTQV (SEQ ID NO: 54), KKRPKP (SEQ ID NO: 55), SSDDE (SEQ ID NO: 56), RRASS (SEQ ID NO: 57), (YXXL)2 (SEQ ID NO: 58), LPLTG (SEQ ID NO: 59), LAFTG (SEQ ID NO: 60), or a combination thereof.

In various embodiments, the targeting domain is a cell-type specific targeting domain. In various embodiments, the cell-type specific targeting domain is or includes an amino acid sequence that includes ASSLNIA (SEQ ID NO:61), KKEEEKKEEEKKEEE (SEQ ID NO: 62), LIFHKEQ (SEQ ID NO: 63), KFNKPFVFLI (SEQ ID NO: 64), QPEHSST (SEQ ID NO: 65), EYHHYNK (SEQ ID NO: 66), NGR (SEQ ID NO: 67), GEKGEP (SEQ ID NO: 68), KTKKK (SEQ ID NO: 69), KALKKK (SEQ ID NO: 70), KGKKK (SEQ ID NO: 71), CSVTCG (SEQ ID NO: 72), LRE (SEQ ID NO: 73), YKYNLNGRES (SEQ ID NO: 74), YRSL (SEQ ID NO: 75), KGGK7 (SEQ ID NO: 76), KKKQYTSIHHG (SEQ ID NO: 77), KDEL (SEQ ID NO: 78), LADQDYTKTA (SEQ ID NO: 79), DDNN (SEQ ID NO: 139), SAVTTVVN (SEQ ID NO: 140), or a combination thereof.

In various embodiments, the engineered polypeptide includes a poly-arginine domain.

In various embodiments, the engineered polypeptide includes a nuclear internalization signal or a nuclear import machinery binding domain. In various embodiments, the nuclear internalization signal or a nuclear import machinery binding domain is or includes an amino acid sequence that includes KKKYKLK (SEQ ID NO: 80), KKRKLE (SEQ ID NO: 81), TRSK (SEQ ID NO: 82), IHRKRKR (SEQ ID NO: 83), NKRKRK (SEQ ID NO: 84), AEKSKKK (SEQ ID NO: 85), RKSK (SEQ ID NO: 86), KRVK (SEQ ID NO: 87), KRK (SEQ ID NO: 88), LQQTPLHLAVI (SEQ ID NO: 89), R_RPR (SEQ ID NO: 90), PRPR (SEQ ID NO: 91), RPPP (SEQ ID NO: 92), RKKRKGK (SEQ ID NO: 93), PAAKRVKLD (SEQ ID NO: 94), KLK IKRPVK (SEQ ID NO: 95), PKKKRKV (SEQ ID NO: 96), QRKRQK (SEQ ID NO. 97), DSPE (SEQ ID NO: 98), FQVT (SEQ ID NO: 99), QSTEKRG (SEQ ID NO: 100), RQGLID (SEQ ID NO: 101), Cyclic RKKH (SEQ ID NO: 102), or a combination thereof.

In various embodiments, the engineered polypeptide includes a nucleic acid release domain. In various embodiments, the nucleic acid release domain is or includes an amino acid sequence that includes GRKKRRQRRRPQ (SEQ ID NO: 103), KRH (SEQ ID NO: 104), KSVKKRSVSEIQ (SEQ ID NO: 105), NRRKKRAL (SEQ ID NO: 106), KFERQ (SEQ ID NO: 107), VRGP (SEQ ID NO: 108), NKDS (SEQ ID NO: 109), NRDN (SEQ ID NO: 110), ANNR (SEQ ID NO: 111), or a combination thereof.

In various embodiments, the engineered polypeptide includes a stability domain. In various embodiments, the stability domain is or includes an amino acid sequence that includes YTRF (SEQ ID NO: 112), GDAY (SEQ ID NO: 113), LLEE (SEQ ID NO: 114), RKKRRQRRR (SEQ ID NO: 115), YKSL (SEQ ID NO: 116), YENF (SEQ ID NO: 117), FQDL (SEQ ID NO: 118), YIGSR (SEQ ID NO: 119), IKVAV (SEQ ID NO: 120), or a combination thereof.

In various embodiments, the engineered polypeptide includes an oligomerization domain. In various embodiments, the oligomerization domain is selected from LIRERTE (SEQ ID NO: 121), LVEERTQ (SEQ ID NO: 122), IITFTK (SEQ ID NO: 123), TLFNK (SEQ ID NO: 124), PIRTLSK (SEQ ID NO: 125), YGNSPLHRFK (SEQ ID NO: 126), FFQKDR (SEQ ID NO: 127), KSRP (SEQ ID NO: 128), YVM (SEQ ID NO: 129), YMKM (SEQ ID NO: 130), RSSSFG (SEQ ID NO: 131), LKIRGRER (SEQ ID NO: 132), LKIRGRKR (SEQ ID NO: 133), HVIFKKVSR (SEQ ID NO: 134), RGPRV (SEQ ID NO: 135), RANVKHLK (SEQ ID NO: 136), YPKAG (SEQ ID NO: 137), and/or YPRTG (SEQ ID NO: 138), optionally where the oligomerization domain is positioned at the C-terminus of the engineered polypeptide.

In various embodiments, the engineered polypeptide includes a linker, optionally where the linker is a linker according to any one of GRKKRRQRRRPQ (SEQ ID NO: 141), KSVKKRSVSEIQ (SEQ ID NO: 142), NRRKKRAL (SEQ ID NO: 143), KFERQ (SEQ ID NO: 144), VRGP, NKDS, NRDN, ANNR, KRH, HL, RI, ET, GQ, RS, RD, RN, RC, RG, RL, DA, RA, GS, LT, FS, GL, SA, DP, GT, GC, RQ, LS, HA, FV, QH, EA, AL, LY, YL, GF, PS, RE, DP, PI, QS, and/or ND.

In various embodiments, an engineered polypeptide is an engineered polypeptide having at least 80% sequence identity (e.g., at least 80%, 85%, 90%, 95%, 96%, 97%, 98%, 99%, or 100%) sequence identity with an engineered polypeptide selected from Table 1.

TABLE 1

| | | Net | | Mole- | Iso- |
|---|---|---|---|---|---|
| | SEQ | charge | Number | cular | electric |
| Exemplary Mini-nucleosome | ID | at | of | weight | point |
| core protein sequences | NO. | pH7 | residues | (g/mol) | (pH) |
| YKRHRKLREKRHRKLRRRRRLKRHRKKRH | 144 | 22.4 | 35 | 4936.95 | 12.56 |
| RKLREK | | | | | |
| KRHRKYGSSLREKRHRKLRRRRRLKRHRK | 145 | 22.4 | 41 | 5369.33 | 12.56 |
| KRHRKLREGGSK | | | | | |
| KRHRKREGSSYLREKRHRKNDLRRRRRLK | 146 | 21.4 | 45 | 5883.82 | 12.34 |
| RHRKKRHRKLREGGSK | | | | | |
| KKPKKREGSSLREYKRHRKNDLRRRRRLK | 147 | 21.3 | 45 | 5787.77 | 12.26 |
| RHRKKRHRKLREGGSK | | | | | |
| RRLARRGSSLREKRHRKYLRRRRRLKKPK | 148 | 22.2 | 42 | 5376.4 | 12.56 |
| KKRHRKLREGGSK | | | | | |
| KRHRKLREKRHRKLREKRHRKLYKRHRKK | 149 | 21.5 | 37 | 5147.17 | 12.34 |
| RHRKLREK | | | | | |
| KRHRKRILREKRHRKLREARKRHRKLKRH | 150 | 23.5 | 41 | 5643.78 | 12.43 |
| RKYKRHRKLREK | | | | | |
| KRHRKKGKKKKGEKGKKKLKGKKKLRRRR | 151 | 25.1 | 36 | 4670.72 | 12.57 |
| RRRQRRY | | | | | |
| KRHRKYAPAPKGKKKKGEKGKKKLKGKKK | 152 | 27.1 | 44 | 5457.69 | 12.57 |
| LKPKPRRRRRRRQRR | | | | | |
| KRHRKGGSGGKGKKKKGEKGKKKLKGKKK | 153 | 25.1 | 42 | 5057.09 | 12.57 |
| LARRRRRRRQRRY | | | | | |
| KRHRKYLREKRHRKRRRRRRRKRHRKYLR | 154 | 24.3 | 35 | 5233.19 | 12.56 |
| EKRRQRR | | | | | |
| KRHRKKRHRKKRYVKKKRHRKRRRRRRDS | 155 | 21.3 | 31 | 4304.19 | 12.64 |
| LL | | | | | |
| KRHRKKRHRKYQKRVKKKRHRKSSSRRRR | 156 | 21.3 | 36 | 4856.72 | 12.47 |
| RRDSLLY | | | | | |
| KRHRKYKKEEEKKEEEKKEEEKRRRRRRR | 157 | 12.1 | 33 | 4636.27 | 11.47 |
| QRRR | | | | | |
| KRHRKWRKKEEEKKEEEKKEEEKYRIRRR | 158 | 14.1 | 37 | 5248.01 | 11.68 |
| RRRRQRRR | | | | | |
| KRHRKYRGDKRHRKRRRRRKRHRKTPHKK | 159 | 20.4 | 30 | 4127.89 | 12.6 |
| K | | | | | |
| KRHRKFIRGDYKRHRKRRRRRKRHRKLAT | 160 | 20.4 | 34 | 4572.45 | 12.6 |
| PHKKK | | | | | |
| KRHRKRGDKRHRKRRRRRKRHRKGSSRNT | 161 | 22.4 | 37 | 4885.68 | 12.64 |
| PHYQKKKK | | | | | |
| KRHRKRGDKRHRKLKRHRKRRRRKRHRKY | 162 | 22.5 | 34 | 4662.54 | 12.64 |
| TPHKK | | | | | |
| KRHRKRGDKRHRKKRHRKKRHRKYRGDKK | 163 | 19.4 | 31 | 4146.88 | 12.32 |
| TK | | | | | |
| KRHRKRGDKKRKKKKRGDYKKRRRRRKKK | 164 | 21.1 | 32 | 4335.19 | 12.17 |
| PPSY | | | | | |
| KRHRKRKRKRKRRRRRKKKRYASSLNIAK | 165 | 24.1 | 33 | 4471.4 | 12.93 |
| RRRR | | | | | |
| KRKKRKGKRLKRRREKRHRKRASSLNIAY | 166 | 20.1 | 33 | 4218.12 | 12.45 |
| KKKK | | | | | |
| KRKKRRLKRKRKRRRRREKRHRKRRRQRR | 167 | 27.1 | 33 | 4781.8 | 12.81 |
| RKKY | | | | | |
| KRKKRRYKRKRRRRRKRHRKLRERKRRLR | 168 | 24.1 | 32 | 4583.57 | 12.59 |
| EKK | | | | | |
| KRKYNGRKRKRKKRHRKKKKRRRRKRHRK | 169 | 28.2 | 35 | 4913.95 | 12.65 |
| YNGRKKK | | | | | |
| KRHRKYWRHRARSKRHRKKKKKKRKKRKG | 170 | 22.3 | 30 | 4065.94 | 12.68 |
| K | | | | | |
| KRHRKRGDKRHRKKKKYNRRKKRALRKKR | 171 | 22.2 | 32 | 4211.1 | 12.51 |
| KGK | | | | | |
| KKRKRGGKTKKKAKKALKKKKKGKKKKRR | 172 | 28 | 39 | 4704.94 | 12.51 |
| RRKYKAAPKK | | | | | |
| KKKAYPKALKKPKKKKKAYPKALKRRRRR | 173 | 29.1 | 43 | 5481.83 | 12.53 |
| KNRRKKRALKRHRK | | | | | |
| KTRSKKKKKRGDKKKKNRRKKRALYNTQI | 174 | 23.1 | 40 | 4888.96 | 12.14 |
| HKKKKKAAPKK | | | | | |
| KGKKKKGEKGKKKLKGKKKLRRRRRYSPK | 175 | 23 | 35 | 4405.41 | 12.46 |
| KRRQRR | | | | | |
| KRHRKYLREKRHRKLRRRRRLKRHRKKRH | 176 | 22.4 | 36 | 5100.12 | 12.42 |
| RKYLREK | | | | | |
| KRHRKYLREKRHRKYLREKRHRKLKRHRK | 177 | 21.5 | 39 | 5473.52 | 12.09 |
| KRHRKLYREK | | | | | |
| KRHRKYKGKKKKGEKGKKKLKGKKKLRRR | 178 | 25.1 | 36 | 4670.72 | 12.57 |
| RRRRQRR | | | | | |
| KRHRKLREKRHRKRRRRRRRKRHRKLREK | 179 | 24.3 | 35 | 5070.02 | 12.68 |
| RRQRRY | | | | | |
| KRHRKKRHRKKRVKKKRHRKRRRRRRDSY | 180 | 21.3 | 31 | 4304.19 | 12.64 |
| LL | | | | | |
| KRHRKKKEEEKKEEEKKEEEKYRRRRRRR | 181 | 12.1 | 33 | 4304.19 | 11.47 |
| QRRR | | | | | |
| KRHRKYQSKKEEEKKEEEKKEEEKNQRRR | 182 | 12.1 | 37 | 5093.71 | 11.47 |
| RRRRQRRR | | | | | |
| KRHRKYRGDKRHRKRRRRRKRHRKYTPHK | 183 | 20.4 | 31 | 4291.06 | 12.43 |
| KK | | | | | |
| KRHRKRGDKRHRKLKRHRKRRRRKRHRKT | 184 | 22.5 | 34 | 4662.54 | 12.64 |
| PHKKY | | | | | |
| KRHRKRGDKRHRKYKRHRKKRHRKRGDKK | 185 | 19.4 | 31 | 4146.88 | 12.32 |
| TK | | | | | |
| KRHRKYRGDKKRKKKKYRGDKKRRRRRKK | 186 | 21.1 | 34 | 4498.36 | 12.03 |
| KPPSY | | | | | |
| KRHRKYGGSRGDKKRKKKKRGDSSSKKRR | 187 | 21.1 | 39 | 4797.6 | 12.17 |
| RRRKKKPPSY | | | | | |
| KRHRKRKRKRKRRRRRKKKRASSLNIAKR | 188 | 24.1 | 33 | 4471.4 | 12.93 |
| RRRY | | | | | |
| KRKKRKGKRLKRRREYKRHRKRASSLNIA | 189 | 20.1 | 33 | 4218.12 | 12.45 |
| KKKK | | | | | |
| KRKKRRLYKRKRKRRRRREKRHRKRRRQR | 190 | 27.1 | 33 | 4781.8 | 12.81 |
| RRKK | | | | | |
| KRKKRRKRKRRRRRKRHRKLRERKRRLRE | 191 | 24.1 | 32 | 4583.57 | 12.59 |
| KKY | | | | | |
| YKRKNGRKRKRKKRHRKKKKRRRRKRHRK | 192 | 28.2 | 35 | 4750.7 | 12.87 |
| NGRKKK | | | | | |
| KRKYWRNGRKRKRQKRHRKKKKRARRRRK | 193 | 30.3 | 41 | 5585.71 | 12.93 |
| RHRKNGRKHKKK | | | | | |
| KRHRKYWRHRARSKRHRKKKPKKRKKRKG | 194 | 21.3 | 30 | 4034.88 | 12.68 |
| K | | | | | |
| KRHRKPKPRIWRHRARSRDYKRHRKKKPK | 195 | 23.3 | 37 | 4897.9 | 12.56 |
| KRKKRKGK | | | | | |

The present disclosure further includes a composition including: (i) at least one sulfated polypeptide agent of the present disclosure, and (11) at least one engineered polypeptide of the present disclosure. In certain embodiments, such a composition includes at least two of the engineered polypeptides, where a first of the engineered polypeptides is able to oligomerize with a second of the engineered polypeptides. In various embodiments, the ratio of the polynucleotides to the engineered polypeptides is between 1:3 and 1:2,000. In various embodiments, the ratio of the polynucleotides to the engineered polypeptides is between 1:3 and 1:1,000, between 1:3 and 1:500, between 1:3 and 1:200, between 1:3 and 1:100, or between 1:3 and 1:50. In various embodiments, the ratio of the polynucleotides to the engineered polypeptides is between 1:200 and 1:2,000, between 1:200 and 1:1000, or between 1:200 and 1:500. In various embodiments, the composition includes a pharmaceutical carrier. The present disclosure further includes a cell including composition including an engineered polypeptide as disclosed herein and a sulfated polypeptide agent as disclosed herein.

In various embodiments, the present disclosure includes a method of condensing a sulfated polypeptide agent of the present disclosure by contacting the expression vector with an engineered polypeptide as disclosed herein. In various embodiments, the present disclosure includes a method of neutralizing the charge of a sulfated polypeptide agent that includes contacting the polynucleotide with an engineered polypeptide as disclosed herein.

### Pharmaceutical Compositions and Administration

The present disclosure contemplates a sulfated polypeptide agent composition that includes a sulfated polypeptide agent and at least one delivery agent and/or at least one carrier. As disclosed herein, a sulfated polypeptide agent composition may be in any form known in the art. A sulfated polypeptide agent composition can be formulated such that it is suitable for laboratory, research, or therapeutic administration to a cell, system, or subject in vitro, ex vivo, or in vivo. A sulfated polypeptide agent composition can be formulated such that it is suitable (e.g., pharmaceutically acceptable) for administration to animals. In various embodiments, a sulfated polypeptide agent composition disclosed herein can be administered, or formulated for administration, to animals of commercial interest, livestock, and household pets such as dogs and cats. In various embodiments, a sulfated polypeptide agent composition disclosed herein can be administered, or formulated for administration, to an animal that is a mammal, e.g., a human.

In various embodiments, a sulfated polypeptide agent composition is a pharmaceutical composition that includes a pharmaceutically acceptable carrier. Formulations that include pharmaceutically acceptable carriers are well known to those skilled in the art, as is the development of suitable dosing and treatment regimens. Selection or use of any particular form may depend, in part, on the intended mode of administration and therapeutic application.

A sulfated polypeptide agent composition can include one or more pharmaceutically acceptable carriers such as one or more solvents, dispersion media, coatings, antibacterial and antifungal agents, isotonic and absorption delaying agents, and the like that are physiologically compatible. Compositions of the present disclosure can include a pharmaceutically acceptable salt, e.g., an acid addition salt or a base addition salt.

In certain embodiments, a sulfated polypeptide agent composition can be administered, or formulated for administration, by subcutaneous, intraocular, intravitreal, parenteral, intravenous, intramuscular, intrathecal, topical, oral, intraperitoneal injections, or nasal inhalation administration. Solutions including expression vectors can be prepared in sterile water or sterile saline and can also be suitably mixed with one or more surfactants, such as pluronic acid. Dispersions may also be prepared in glycerol, liquid polyethylene glycols, and mixtures thereof. Storage preparations may contain preservatives to prevent microorganisms from growing.

In various embodiments, a sulfated polypeptide agent composition can be administered, or formulated for administration, as a sterile formulation for injection, and/or can be formulated in accordance with conventional pharmaceutical practices using distilled water for injection as a vehicle. Expression vector compositions suitable for injection can include sterile aqueous solutions or dispersions. Expression vector compositions suitable for injection can include physiological saline or an isotonic solution containing glucose and other supplements such as D-sorbitol, D-mannose, D-mannitol, and sodium chloride may be used as an aqueous solution for injection, optionally in combination with a suitable solubilizing agent, for example, alcohol such as ethanol and polyalcohol such as propylene glycol or polyethylene glycol, and a nonionic surfactant such as polysorbate 80™, HCO-50 and the like.

A formulation can be sterile and must be fluid to allow proper flow in and out of a syringe. A formulation can also be stable under the conditions of manufacture and storage. A carrier can be a solvent or dispersion medium containing, for example, water and saline or buffered aqueous solutions. Preferably, isotonic agents, for example, sugars or sodium chloride can be used in the formulations. For human administration, final preparations and compositions should meet sterility, pyrogenicity, and the general endotoxin levels, safety and purity standards as required by the US FDA and EU regulatory standards. Temperature and exposure to other proteins can alter the properties of formulations.

In various embodiments, a sulfated polypeptide agent composition of the present disclosure can be administered, or formulated for administration, as a solution, microemulsion, dispersion, liposome, or other ordered structure suitable for stable storage at high concentration. Sterile injectable solutions can be prepared by incorporating a composition disclosed herein in the required amount in an appropriate solvent with one or a combination of ingredients enumerated above, as required, followed by filter sterilization. Generally, dispersions are prepared by incorporating a composition disclosed herein into a sterile vehicle that contains a basic dispersion medium together with any other required ingredients known in the art. In the case of sterile powders for the preparation of sterile injectable solutions, methods for preparation include vacuum drying and freeze-drying that yield a powder of a composition disclosed herein plus any additional desired ingredient (see below) from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts, and gelatin.

A sulfated polypeptide agent composition as disclosed herein can be administered, or formulated for administration, as an immunoliposome composition. Such formulations can be prepared by methods known in the art.

In some embodiments, a sulfated polypeptide agent composition can be administered, or formulated for administration, by intrapulmonary administration (e.g., administration via an inhaler or nebulizer) to a mammal such as a human. Methods for formulating such compositions are well known in the art. Dry powder inhaler formulations and suitable systems for administration of the formulations are also known in the art. Pulmonary administration may be oral and/or nasal. Examples of pharmaceutical devices for pulmonary delivery include metered dose inhalers, dry powder inhalers (DPIs), and nebulizers. For example, a composition disclosed herein can be administered to the lungs of a subject by way of a dry powder inhaler. In some embodiments, a composition disclosed herein can be intrapulmonarily administered by way of a metered dose inhaler.

In some embodiments, a sulfated polypeptide agent composition can be administered, or formulated for administration, to the eye, e.g., in the form of a pharmaceutically acceptable solution, suspension or ointment. A preparation for use in treating an eye can be in the form of a sterile aqueous solution containing, e.g., additional ingredients such as, but not limited to, preservatives, buffers, tonicity agents, antioxidants and stabilizers, nonionic wetting or clarifying agents, and viscosity-increasing agents. A composition as disclosed herein can be administered topically to the eye of the subject in need of treatment (e.g., a subject afflicted with AMD) by conventional methods, e.g., in the form of drops, or by bathing the eye in a therapeutic solution, containing one or more compositions. A variety of devices for introducing drugs into the vitreal cavity of the eye may be appropriate, in certain embodiments, for administration of a composition as disclosed herein.

Expression vector compositions can be administered, or formulated for administration, by systemic or local delivery can be in the form of injectable or infusible solutions. A sulfated polypeptide agent composition can be administered, or formulated for administration, by a parenteral mode (e.g., intravenous, subcutaneous, intraperitoneal, or intramuscular injection). As used herein, parenteral administration refers to modes of administration other than enteral and topical administration, usually by injection, and include, without limitation, injection, intravenous, intranasal, transnasal, intraocular, pulmonary, intramuscular, intraarterial, intrathecal, intracapsular, intraorbital, intracardiac, intradermal, intrapulmonary, transpulmonary, intraperitoneal, transtracheal, subcutaneous, transcutaneous, subcuticular, intraarticular, subcapsular, subarachnoid, intraspinal, epidural, intracerebral, intracranial, intracarotid and intrasternal injection and infusion. Administration can be systemic or local by intravenous injection, intramuscular injection, intraperitoneal injection, subcutaneous injection.

Expression vector compositions can be administered, or formulated for administration, parenterally, e.g., in the form of an injectable formulation comprising a sterile solution or suspension in water or another pharmaceutically acceptable liquid. For example, a sulfated polypeptide agent composition can be formulated by suitably combining expression vector with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. Nonlimiting examples of oily liquid include sesame oil and soybean oil, and it may be combined with benzyl benzoate or benzyl alcohol as a solubilizing agent. Expression vector compositions can further include a buffer such as a phosphate buffer, or sodium acetate buffer, a soothing agent such as procaine hydrochloride, a stabilizer such as benzyl alcohol or phenol, and an antioxidant. A sulfated polypeptide agent composition formulated for injection can be packaged in a suitable ampule.

Expression vector compositions can be administered, or formulated for administration, via electroporation, sonophoresis, intraosseous injections methods or by using gene gun. Expression vectors may also be implanted into microchips, nano-chips, or nanoparticles.

Expression vector compositions can be administered, or formulated for administration, by way of local administration. As used herein, “local administration” or “local delivery,” can refer to delivery that does not rely upon transport of the expression vector composition or expression vector composition to its intended target tissue or site via the vascular system. For example, a sulfated polypeptide agent composition may be delivered by injection or implantation of a sulfated polypeptide agent composition or by injection or implantation of a device containing the composition. In certain embodiments, following local administration in the vicinity of a target tissue or site, the composition, or one or more components thereof, may diffuse to an intended target tissue or site that is not the site of administration.

Expression vector compositions can be administered, or formulated for administration, by subcutaneous administration, e.g., accomplished by means of a device, such as a syringe, a prefilled syringe, an auto-injector (e.g., disposable or reusable), a pen injector, a patch injector, a wearable injector, an ambulatory syringe infusion pump with subcutaneous infusion sets, or other device for subcutaneous injection.

A suitable means of administration of a sulfated polypeptide agent composition can be selected based on the condition or disease to be treated and/or upon the age and condition of a subject. A sulfated polypeptide agent can be administered to a subject either alone or in combination with one or more other therapeutic modalities.

Dose and method of administration can vary depending on the weight, age, condition, and the like of a patient, and can be suitably selected as needed by those skilled in the art. Other factors such as solubility, bioavailability, half-life, shelf-life will be contemplated by one skilled in the art. A suitable dose of a sulfated polypeptide agent composition disclosed herein, which dose is capable of treating or preventing a disorder in a subject, can depend on a variety of factors including, e.g., the age, sex, and weight of a subject to be treated, the condition or disease to be treated, and the particular expression vector composition used. Other factors affecting the dose administered to the subject include, e.g., the type or severity of the condition or disease. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject can also be adjusted based upon the judgment of a medical practitioner.

A sulfated polypeptide agent composition solution can include a therapeutically effective amount of a composition disclosed herein. Such effective amounts can be readily determined by one of ordinary skill in the art based, in part, on the effect of the administered composition, or the combinatorial effect of the composition and one or more additional active agents, if more than one agent is used. A therapeutically effective amount can be an amount at which any toxic or detrimental effects of the composition are outweighed by therapeutically beneficial effects.

In various embodiments, expression vector composition of the present disclosure could be used to deliver nucleotides to one or more tissues or cell types selected from liver, retina, skeletal muscle, and neurons or the central nervous system. In various embodiments, a sulfated polypeptide agent composition can include 10² copies or more (e.g., at least 10², 10⁴, 10⁶, 10¹, 10¹⁰, 10¹², 10¹⁴, or 10¹⁶ copies) of a desired transgene.

In some embodiments, the compositions provided herein are present in unit dosage form, which unit dosage form can be suitable for self-administration. Such a unit dosage form may be provided within a container, typically, for example, a vial, cartridge, prefilled syringe or disposable pen. A doser may also be used, for example, with an injection system as disclosed herein.

In some embodiments, a sulfated polypeptide agent composition can be formulated for storage at a temperature below 0° C. (e.g., −20° C. or −80° C.). In some embodiments, the composition can be formulated for storage for up to 2 years (e.g., one month, two months, three months, four months, five months, six months, seven months, eight months, nine months, 10 months, 11 months, 1 year, 1½ years, or 2 years) at 2-8° C. (e.g., 4° C.). Thus, in some embodiments, the compositions disclosed herein are stable in storage for at least 1 year at 2-8° C. (e.g., 4° C.). In particular instances, a sulfated polypeptide agent composition can be formulated as a solution. In some embodiments, a composition can be formulated, for example, as a buffered solution at a suitable concentration and suitable for storage at 2-8° C. (e.g., 4° C.). In some embodiments, a sulfated polypeptide agent composition can be formulated for storage at room temperature (20-25 degree Celsius).

In certain embodiments, compositions can be formulated with a carrier that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for the preparation of such formulations are known in the art.

### Examples

The present Example(s) demonstrate that systemic (e.g., intravenous) delivery of sulfated polypeptide agents achieves delivery and/or increased delivery of the polypeptide agent to muscle. The present Examples represent that a variety of polypeptide agents can be advantageously provided in a sulfated form for systemic administration, such polypeptide agents including for example antibodies, antibody fragments, lipid nanoparticles (e.g., lipid nanoparticles modified with peptides that can be sulfated polypeptides such as sulfated targeting polypeptides), and viral vectors (e.g., AAV and lentiviral vectors), e.g., where the sulfated polypeptide agent achieves one or more of enhanced delivery to or transduction of target cells, tissues, or organs (e.g., muscle), increased liver escape, reduced liver accumulation. The present Examples also provide, among other things, muscle specific promoters than drive enhanced levels of expression compared to certain promoters commercially utilized for expressing coding sequences in muscle. Those of skill in the art will appreciate from the present Examples that exemplified polypeptides (e.g., sulfated polypeptides) and nucleic acids, and their uses, are representative of polypeptides, nucleic acids, and uses of the present disclosure and demonstrate that principle that sulfated polypeptides disclosed herein can be associated with and/or used to deliver nucleic acids, e.g., when a sulfated polypeptide and a nucleic acid are associated (which associated combination can in some embodiments of the present disclosure be referred to as a mini-nucleosome).

### Example 1: Improved Delivery of Sulfated Polypeptide Agents to Muscle Cells and Tissues

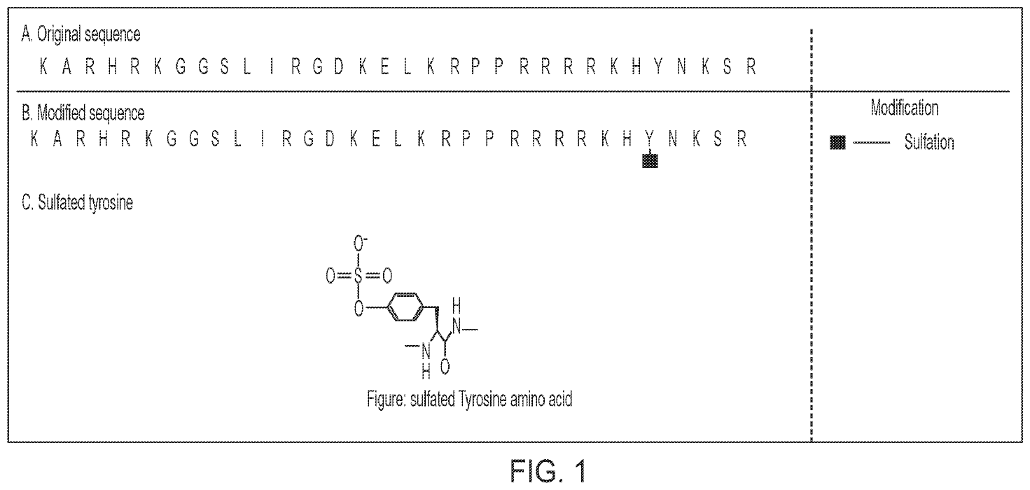

The present Example demonstrates that a polypeptide agent that is a vector for delivery of associated nucleic acids can achieve delivery to and/or increased delivery to muscle as a sulfated polypeptide as compared to a non-sulfated reference polypeptide agent. In particular, the present disclosure compares systemic administration of a sulfated and non-sulfated forms of a polypeptide agent that is a vector for nucleic acid delivery, which forms are depicted in FIG. 1. The sulfated polypeptide agent of this Example is referred to herein as Agent 118, while the non-sulfated polypeptide agent of this Example is referred to as Agent 117.

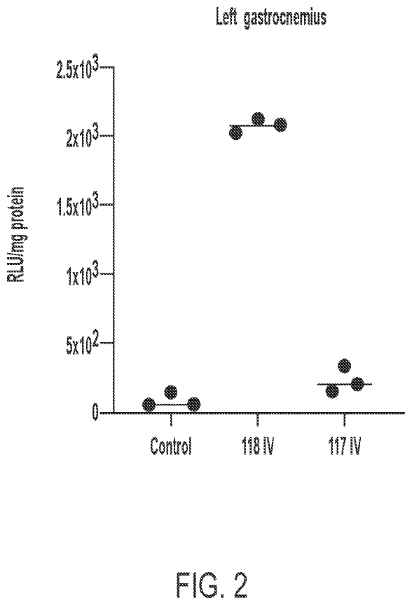

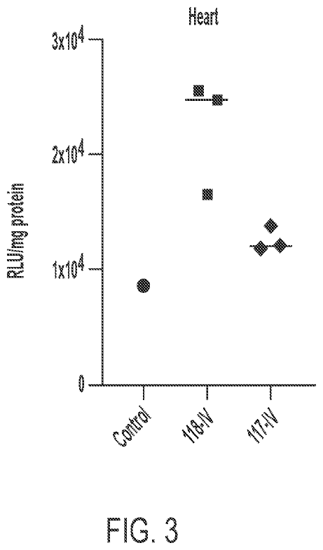

Balb/c mice were injected intravenous (via tail vein) with 118-Luc or 117-Luc (1.8e13/ml) (n=3 mice/group; n=1 for untreated control muscle). 25 days following IV injection, tissues were collected and snap-frozen following euthanasia and intracardiac perfusion with PBS. Frozen tissues were lysed in Glo-lysis buffer. Equal amounts of ONE-Glo™ Luciferase Assay System (E6110, Promega) were added to the lysed tissues and luminescence was measured in a plate reader. Values are depicted relative to the amount of protein in each sample as measured using BCA assay. Results shown for the left gastrocnemius (FIG. 2) and for the heart (FIG. 3) demonstrate enhanced luciferase expression from a sulfated polypeptide agent as compared to a non-sulfated form of the same polypeptide agent in mouse hind limbs and heart following intravenous injection.

### Example 2: Sulfated Polypeptide Agent can Efficiently Deliver a Therapeutic Nucleic Acid Payload to Target Muscle Cells

The present Example demonstrates that a polypeptide agent that is a vector for delivery of associated nucleic acids can achieve delivery to and/or increased delivery to muscle as a sulfated polypeptide agent as compared to a non-sulfated reference polypeptide agent. In particular, the present disclosure compares systemic administration of Agent 117 and Agent 118 associated with nucleic acid molecules encoding dystrophin, a model therapeutic agent for treatment of Duchenne Muscular dystrophy (DMD).

DMD is an X-linked progressive muscle disorder caused by mutation in the gene encoding dystrophin (DMD), a muscle protein important for muscle integrity and strength. The dystrophin cDNA is ˜11 kb and thus cannot be packaged in AAV, a leading technology for gene therapy, due to its packaging capacity constraints. The present Example demonstrates compositions and methods for the delivery of nucleic acids that encode the full length dystrophin protein to restore expression in skeletal muscles and/or heart and/or diaphragm. In this Example, mini-nucleosome core proteins associated with one or multiple polypeptides condense the nucleic acids into small particles.

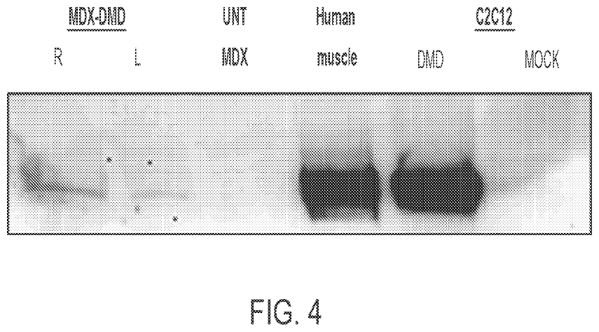

Mdx mice were injected intramuscular into the gastrocnemius muscle with the sulfated form of the mini-nucleosome polypeptide agent associated with nucleic acids encoding DMD at a dose of 1e13/ml (n=1; MDX-DMD: R=right leg and L=left leg). 4 days following IM injection, muscles tissues were collected and snap-frozen following intracardiac perfusion with PBS. Tissues were lysed in RIPA buffer, 60 ug was loaded on a 3-8% Tris-Acetate gel. Western blot was performed and human dystrophin was visualized with a mouse anti-human dystrophin antibody. Controls include muscle lysates from untreated mdx (UNT MDX) mice, human muscle lysates and lysates from mouse C2C12 myoblast cells transfected with the DMD-plasmid (C2C12 DMD) or mock-transfected (C2C12 MOCK). Results, as shown in FIG. 4, show expression of full length human dystrophin following intramuscular injection of mini-nucleosome expressing human dystrophin (DMD).

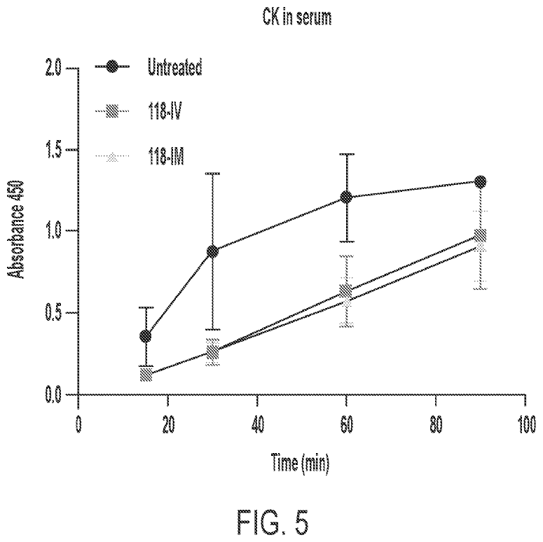

CK levels were also measured in treated and untreated Mdx mice. Mdx mice were injected intravenously (via tail vein) or intramuscularly (gastrocnemius muscle) with saline or sulfated-mini-nucleosome associated with nucleic acids encoding DMD (1.5e13/ml); 117-14893 (1.8e13/ml) (n=3 mice/group). Blood was collected via cardiac puncture and serum was prepared following a 6 min spin at 13000 rpm. CK values were determined using a colorimetric assay from Abcam. Untreated Mdx mice (n=2) were used as control. Results are shown in FIG. 5.

### Example 3: Expression from Muscle Promoters

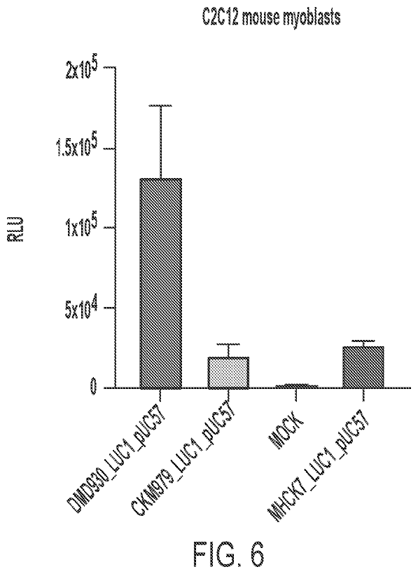

The present Example includes the recognition that certain promoters utilized herein have advantageous characteristics for expression of operably linked coding sequences in muscle cells, as exemplified in the present Example in C2C12 mouse myoblasts as a representative muscle cell type. In the present Example, a luciferase reporter construct was expressed in C2C12 mouse myoblasts using each of three promoters. Cells were mock transfected or transfected with vectors expressing luciferase using Lipofectamine LTS Plus reagent in a 96-well plate. 48 hours post transfection luciferase activity was measured using ONE-Glo™ Luciferase Assay System (E6110, Promega). Data shown in FIG. 6 demonstrate that the promoter utilized in the luciferase expression construct of the first column (a promoter according to SEQ ID NO: 3) provided advantageous expression in the representative muscle cells, and further demonstrated substantially greater expression as compared to the promoters utilized in the luciferase expression construct of the second column (a promoter according to SEQ ID NO: 4) and fourth column (a promoter according to SEQ ID NO: 5), as well as the mock control of the third column.

### Example 4: Expression from Nucleic Acids Associated with Sulfated Polypeptide Agents

The present Example demonstrates that a polypeptide agent that is a vector for delivery of associated nucleic acids can achieve delivery and expression of the nucleic acids in muscle. The present Example specifically demonstrates expression in the tibialis anterior and gastrocnemius muscles of a polypeptide (luciferase) encoded by a nucleic acid intravenously injected into mice in association with a sulfated polypeptide (an intravenously administered sulfated mini-nucleosome referred to in this Example as 118 IV).

In this Example, sulfated polypeptides associated with nucleic acids encoding luciferase were injected intravenously (via tail vein) into Mdx mice (2.2e13/ml) every week for 3 weeks (injection on days 1, 8, and 15). Tissue samples were collected and snap-frozen 28 days after the first injection of 118 IV associated with the nucleic acid encoding luciferase, following euthanasia and intracardiac perfusion with PBS. Frozen tissues were lysed in Glo-lysis buffer. Equal amounts of ONE-Glo™ Luciferase Assay System (E6110, Promega) were added to the lysed tissues and luminescence was measured in a plate reader.

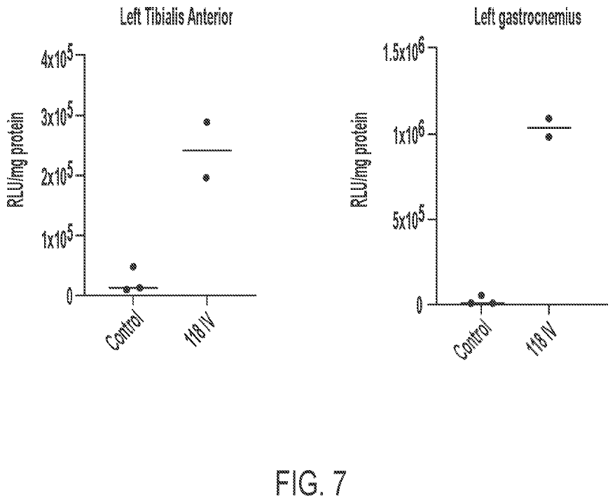

Luciferase expression was greater when the nucleic acid encoding the luciferase was administered in conjunction with the sulfated 118 IV polypeptide, as compared to luciferase expression in untreated mice (control). Data are shown in FIG. 7. Values are depicted relative to the amount of protein in each sample as measured using BCA assay.

### Example 5: Sulfated Polypeptide Agent can Efficiently Deliver a Therapeutic Nucleic Acid Payload to Target Muscle Cells by Intravenous Injection

The present Example demonstrates that a polypeptide agent that is a vector for delivery of associated nucleic acids can achieve delivery to and/or increased delivery to muscle as a sulfated polypeptide agent as compared to a non-sulfated reference polypeptide agent when administered intravenously. In particular, the present disclosure shows that systemic administration of a sulfated polypeptide agent according to the present disclosure (118) associated with nucleic acid molecules encoding dystrophin to a mouse model of DMID dramatically decreased creatine kinase (CK) levels and unexpectedly maintained a decrease in CK levels for at least 7.5 months. Decreased CK levels are indicative of successful treatment of DMD.

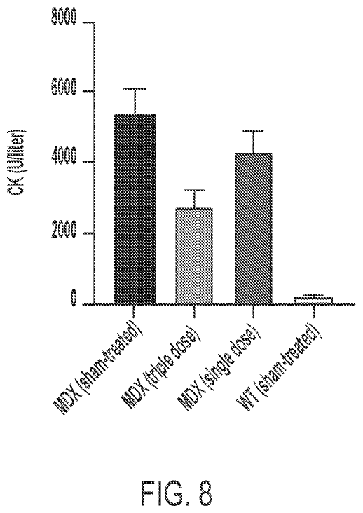

In this Example, sham-treated wt and MDX mice were injected intravenously (via tail vein) every week for 3 weeks (injection on days 1, 8, and 15) with sulfated polypeptides expressed from nucleic acids encoding GFP and luciferase, respectively. MDX mice were injected intravenously (via tail vein) with Agent 118 associated with a plasmid encoding dystrophin (plasmid 14893; together 118-14893). MDX mice were injected intravenously with a single dose of 118-14893 on day 1 of the experiment (single dose group), or with three doses of 118-14893 delivered on days 1, 8, and 15 of the experiment (triple dose group), each injection at 2.2 e13/ml particles (n=5-6/group). CK levels were measured in serum collected 28 days post injection for mice of the single dose group and 14 days post injection for mice of the triple dose group. Sham-treated MDX (n=5) and WT mice (n=3) were included as controls. Results shown in FIG. 8 show that all treatments, but not controls, decreased CK levels dramatically.

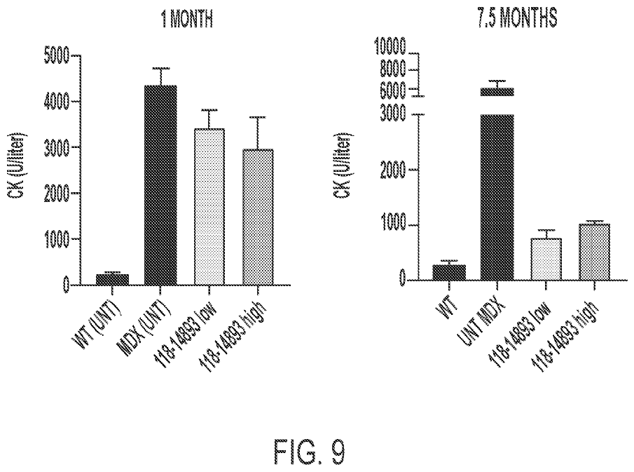

This Example further includes data showing that the advantages of agents disclosed in the present Example and application are demonstrated across dosages. MDX mice were injected intravenously with a single dose of 118-14893 at 2e12/ml (low) or 8e12/ml (high) particles. CK levels were measured 1 month post injections and 7.5 months post injection from serum. Untreated MDX (UNT MDX) and wild type (WT) mice were included as controls (n=3-6/group). Data is not shown for use of an unsulfated from of 118 because no significant expression of dystrophin was found to occur, and accordingly no effect on CK levels would result. As shown in FIG. 9, both doses dramatically decreased CK levels and unexpectedly maintained a decrease in CK levels for at least 7.5 months post injection.

In this Example, blood was collected from mice via cardiac puncture and serum was prepared following a 6 min spin at 13000 rpm. CK values were measured in using the Stanbio CK-MB Liqui-UV kit according to the manufacturer's instructions.

### Example 6: Dystrophin is Expressed in Muscle after Intravenous Injection of Therapeutic Nucleic Acid Associated with a Sulfated Polypeptide Agent

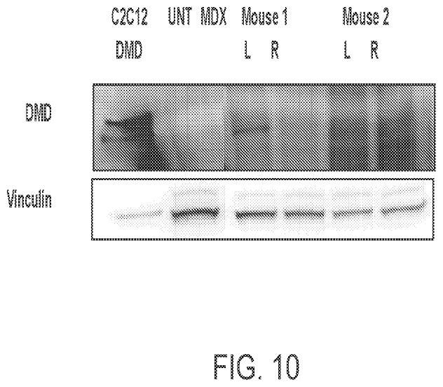

The present Example confirms that dystrophin is expressed in mice following intravenous injection of a nucleic acid encoding dystrophin associated with a sulfated polypeptide of the present disclosure. In particular, MDX mice were injected intravenously (via tail vain) with a nucleic acid encoding dystrophin in association with representative sulfated polypeptide 118 (together 118-DMD, delivered at 8e12/ml). Hindlimb muscles tissues were collected from the mice two months or three months post intravenous injection (for mice 1 and 2 respectively) and snap-frozen following intracardiac perfusion with PBS. Tissues were lysed in RIPA buffer and 60 ug was loaded on a 3-8% Tris-Acetate gel. Western blot was performed and human dystrophin was visualized with a mouse anti-human dystrophin antibody. Muscle lysates from an untreated MDX mouse were included as a negative control. Cell lysates from C2C12 mouse myoblast cell lines transfected with an expression plasmid encoding dystrophin (14893 plasmid) were included as a positive control. Vinculin was used as loading control. FIG. 10 shows expression of full length human dystrophin in mice of this Example as described, following intravenous injection of the 118-DMD mini-nucleosome.

SELECTED SEQUENCES
SEQ ID NO.: 3
CAGGCCCTGTAACTTTTCCAATAAAATATTCTATTAGAAACAAGTTCCTTCTAGATCTTCCTAA
AAGAATACTAAAGGACCAAACCGTGCAACAGCACTGGTGGTATTGTCTTAATTTAGAGAGTCCA
TACCAGTGATTCTCAAACATTAGTATGCTCAGAGTTTCTGGTTCAATAGGTCTGGGCCGGGCCC
AGGGAATTTTCATTTCTAAGAAGTTCGCAGGTGATACTGATAACTGCTGTTCCCAGGGTCTACA
TTTTGAGATCCGCTGCTATGTCACAAATCCAAATCCAAAGTCTTTTTAACAATCCCTTTTAGCC
CAGGATGAGCCTAACGGCAATTTCACGTCCTTATCGCCGGCCACTTTAATCTTCAACTGAGGAG
GAAGGGAGAGTGAGAATTATGTATATAATTTTTTTCTTTTATTTTTTAAAAAGAAAACTCCAAG
CCCACACTCTTAATTATCTGGGATGAGGTCATTTGCACCTGGGTCATTTATTTAGACAACAAGT
AGTCTTGCTAGAGGCTTTAAGGAGGTGTTAGCAGAGCGGTGTGTGGCTTCCAGGTTCCTGGGTC
TACCTTAAATCCCCATCCTCTTTCTTTCTTTCTTTCTTTCTTTCTTTCTCTTTTCTTTTTCCTT
TCCGTTTTGAAAGCCCCAACTGCCCAGTCTCTGGGTGTCCGGGTTCCCAGGTCCCTAGGTGGGG
GCGGAGCCCGACACGGCCCCGCCCCGTCTGCCCGCAGCGCCCCCTCCCCGGCCCGCCCGCGCCG
GCTCCTCCGCAGTGCTTTCAGCTGTGAGCTTGGGCGGCGGCGGCGGCGGCGCTCCACTTTCGGG
GAGCCCGGCGGCTCTGGGAAGCTCACTCCTCCACTCGTACCCACACTCGACCGCGGAGCCCTTG
CAGCCATGAGGGAACAGCTCAAAGGGTAAGTGGA
SEQ ID NO.: 4
CCGGTTATAATTAACCAGGACACGTGGCGAACCCCCCTCCAACACCTGCCCCCGAACCCCCCCA
TACCCAGCGCCTCGGGTCTCGGCCTTTGCGGCAGAGGAGACAGCAAAGCGCCCTCTAAAAATAA
CTCCTTTCCCGGCGACCGAGACCCTCCCTGTCCCCCGCACAGCGGAAATCTCCCAGTGGCACCG
AGGGGGCGAGGGTTAAGTGGGGGGGAGGGTGACCACCGCCTCCCACCCTTGCCCTGAGTTTGAA
TCTCTCCAACTCAGCCAGCCTCAGTTTCCCCTCCACTCAGTCCCTAGGAGGAAGGGGCGCCCAA
GCGCGGGTTTCTGGGGTTAGACTGCCCTCCATTGCAATTGGTCCTTCTCCCGGCCTCTGCTTCC
TCCAGCTCACAGGGTATCTGCTCCTCCTGGAGCCACACCTTGGTTCCCCGAGGTGCCGCTGGGA
CTCGGGTAGGGGTGAGGGCCCAGGGGGCACAGGGGGAGCCGAGGGCCACAGGAAGGGCTGGTGG
CTGAAGGAGACTCAGGGGCCAGGGGACGGTGGCTTCTACGTGCTTGGGACGTTCCCAGCCACCG
TCCCATGTTCCCGGCGGGGGGCCAGCTGTCCCCACCGCCAGCCCAACTCAGCACTTGGTCAGGG
TATCAGCTTGGTGGGGGGGCGTGAGCCCAGCCCCTGGGGCGGCTCAGCCCATACAAGGCCATGG
GGCTGGGCGCAAAGCATGCCTGGGTTCAGGGTGGGTATGGTGCGGGAGCAGGGAGGTGAGAGGC
TCAGCTGCCCTCCAGAACTCCTCCCTGGGGACAACCCCTCCCAGCCAATAGCACAGCCTAGGTC
CCCCTATATAAGGCCACGGCTGCTGGCCCTTCCTTTGGGTCAGTGTCACCTCCAGGATACAGAC
AGCCCCCCTTCAGCCCAGCCCAGCCAGGTACTGCACGGGGCGGGAATCTGGGTGGGGGCCAGAG
TAGGGGATTTCTGTGGGTG
SEQ ID NO.: 5
AAGCTTGCATGTCTAAGCTAGACCCTTCAGATTAAAAATAACTGAGGTAAGGGCCTGGGTAGGG
GAGGTGGTGTGAGACGCTCCTGTCTCTCCTCTATCTGCCCATCGGCCCTTTGGGGAGGAGGAAT
GTGCCCAAGGACTAAAAAAAGGCCATGGAGCCAGAGGGGCGAGGGCAACAGACCTTTCATGGGC
AAACCTTGGGGCCCTGCTGTCTAGCATGCCCCACTACGGGTCTAGGCTGCCCATGTAAGGAGGC
AAGGCCTGGGGACACCCGAGATGCCTGGTTATAATTAACCCAGACATGTGGCTGCCCCCCCCCC
CCCAACACCTGCTGCCTCTAAAAATAACCCTGTCCCTGGTGGATCCCCTGCATGCGAAGATCTT
CGAACAAGGCTGTGGGGGACTGAGGGCAGGCTGTAACAGGCTTGGGGGCCAGGGCTTATACGTG
CCTGGGACTCCCAAAGTATTACTGTTCCATGTTCCCGGCGAAGGGCCAGCTGTCCCCCGCCAGC
TAGACTCAGCACTTAGTTTAGGAACCAGTGAGCAAGTCAGCCCTTGGGGCAGCCCATACAAGGC
CATGGGGCTGGGCAAGCTGCACGCCTGGGTCCGGGGTGGGCACGGTGCCCGGGCAACGAGCTGA
AAGCTCATCTGCTCTCAGGGGCCCCTCCCTGGGGACAGCCCCTCCTGGCTAGTCACACCCTGTA
GGCTCCTCTATATAACCCAGGGGCACAGGGGCTGCCCTCATTCTACCACCACCTCCACAGCACA
GACAGACACTCAGGAGCAGCCAGC

### OTHER EMBODIMENTS

It will be appreciated that the scope of the present disclosure is to be defined by that which may be understood from the disclosure and claims rather than by the specific embodiments that have been presented by way of example. Elements described with respect to one aspect or embodiment of the present disclosure are also contemplated with respect to other aspects or embodiments of the present disclosure. For example, elements of claims that depend directly or indirectly from a certain independent claim presented herein serve as support for those elements being presented in additional dependent claims of one or more other independent claims. Throughout the description, where compositions or methods are described as having, including, or comprising specific elements, compositions that consist essentially of, consist of, or do not comprise the recited elements are likewise hereby disclosed. All references cited herein are hereby incorporated by reference.

What is claimed is:

1. A method of delivering a polypeptide agent to one or more target cells, tissues, or organs of a subject, the method comprising systemically delivering a sulfated polypeptide agent.

2. A method of reducing toxicity of a polypeptide agent comprising systemically delivering a sulfated form of the polypeptide agent to a subject, wherein delivery of the sulfated polypeptide agent to, or accumulation of the sulfated polypeptide agent in, liver and/or kidney of the subject is reduced as compared to a reference non-sulfated form of the polypeptide agent.

3. A method of achieving delivery of a polypeptide agent to one or more target cells, tissues, or organs of a subject, the method comprising systemically delivering a sulfated form of the polypeptide agent to a subject, wherein delivery of the sulfated polypeptide agent to, or accumulation of the sulfated polypeptide agent in, the target cells, tissues, or organs of the subject is increased as compared to a reference non-sulfated form of the polypeptide agent.

4. The method of any one of claims 1-3, wherein the sulfated polypeptide agent comprises at least two, three, four, or five sulfated residues.

5. The method of any one of claims 1-4, wherein the sulfated polypeptide comprises a sulfated tyrosine residue.

6. The method of any one of claims 1-5, where in the sulfated polypeptide agent is an antibody or antibody fragment.

7. The method of any one of claims 1-5, wherein the sulfated polypeptide agent is an enzyme, structural protein, or viral vector.

8. The method of any one of claims 1-5, wherein the sulfated polypeptide agent is an engineered polypeptide comprising (i) a nucleic acid binding domain and (ii) at least one of a nucleic acid release domain, a nuclear localization signal, a stability domain, an oligomerization domain, and a targeting domain, optionally wherein the sulfated polypeptide is associated with one or more nucleic acid sequences.

9. The method of claim 8, wherein the sulfated polypeptide agent is a mini-nucleosome core protein.

10. The method of any one of claims 1-5, wherein the sulfated polypeptide is a sulfated form of a polypeptide agent of which a non-sulfated form has been approved by the United States Food and Drug Administration (FDA) for administration to humans.

11. The method of any one of claims 1-10, wherein the sulfated polypeptide agent is a therapeutic agent.

12. The method of claim 1-11, wherein the target cell, tissue, or organ is muscle tissue.

13. The method of any one of claims 1-12, wherein the target cell, tissue, or organ is skeletal muscle, cardiac muscle, diaphragm, or a combination thereof.

14. The method of any one of claims 1-13, wherein the systemic delivery comprises intravenous delivery.

15. The method of any one of claims 1-14, wherein the method is characterized by increased delivery of the polypeptide agent to, or increased accumulation of the polypeptide agent in, the target cells, tissues, or organs as compared to a reference non-sulfated form of the polypeptide agent.

16. The method of any one of claims 1-15, wherein the method is characterized by reduced delivery of the polypeptide agent to, or reduced accumulation of the polypeptide agent in, liver and/or kidney as compared to a reference non-sulfated form of the polypeptide agent.

17. The method of any one of claims 1-16, wherein the sulfated polypeptide is associated with one or more nucleic acid sequences encoding one or more expression products and delivery results in expression of the expression products, optionally wherein expression of the expression products persists for at least 1 week, 2 weeks, 3 weeks, 1 month, 2 months, 3 months, 4 months, 5 months, 6 months, 7 months, 8 months, 10 months, 11 months, or 1 year after delivery.

18. A pharmaceutical composition comprising a sulfated polypeptide agent, wherein the composition is formulated for systemic delivery of the sulfated polypeptide to a subject.

19. The pharmaceutical composition of claim 18, wherein the sulfated polypeptide agent comprises at least two, three, four, or five sulfated residues.

20. The pharmaceutical composition of claim 18 or 19, wherein the sulfated polypeptide comprises a sulfated tyrosine residue.

21. The pharmaceutical composition of any one of claims 18-20, where in the sulfated polypeptide agent is an antibody or antibody fragment.

22. The pharmaceutical composition of any one of claims 18-20, wherein the sulfated polypeptide agent is an enzyme, structural protein, or viral vector.

23. The pharmaceutical composition of any one of claims 18-20, wherein the sulfated polypeptide agent is an engineered polypeptide comprising (i) a nucleic acid binding domain and (ii) at least one of a nucleic acid release domain, a nuclear localization signal, a stability domain, an oligomerization domain, and a targeting domain, optionally wherein the sulfated polypeptide is associated with one or more nucleic acid sequences.

24. The pharmaceutical composition of claim 23, wherein the sulfated polypeptide agent is a mini-nucleosome core protein.

25. The pharmaceutical composition of any one of claims 18-20, wherein the sulfated polypeptide is a sulfated form of a polypeptide agent of which a non-sulfated form has been approved by the United States Food and Drug Administration (FDA) for administration to humans.

26. The pharmaceutical composition of any one of claims 18-25, wherein the sulfated polypeptide agent is a therapeutic agent.

27. The pharmaceutical composition of any one of claims 18-26, wherein the systemic delivery comprises intravenous delivery.

28. The pharmaceutical composition of any one of claims 18-27, wherein the pharmaceutical composition achieves increased delivery of the polypeptide agent to, or increased accumulation of the polypeptide agent in, the target cells, tissues, or organs as compared to a reference pharmaceutical composition comprising a non-sulfated form of the polypeptide agent.

29. The pharmaceutical composition of any one of claims 1-28, wherein the pharmaceutical composition achieves reduced delivery of the polypeptide agent to, or reduced accumulation of the polypeptide agent in, liver and/or kidney as compared to a reference pharmaceutical composition comprising a non-sulfated form of the polypeptide agent.

30. A method of producing a nucleic acid vector for expression of an encoded polypeptide in muscle, the method comprising operably linking a promoter nucleic acid sequence having at least 80% sequence identity with SEQ ID NO: 3 with a coding sequence.

31. A method of expressing a polypeptide in muscle, the method comprising administering to a cell, system, or subject a nucleic acid comprising a promoter nucleic acid sequence having at least 80% sequence identity with SEQ ID NO: 3 operably linked with a coding sequence encoding the polypeptide.

32. A nucleic acid comprising a promoter nucleic acid sequence having at least 80% sequence identity with SEQ ID NO: 3 operably linked with a coding sequence encoding a polypeptide for expression in muscle.

33. A method or composition of any one of claims 1-32, wherein the sulfated polypeptide agent does not comprise (i) a nucleic acid binding domain and/or (ii) at least one of a nucleic acid release domain, a nuclear localization signal, a stability domain, an oligomerization domain, and a targeting domain

34. A method or composition of any one of claims 1-33, wherein the sulfated polypeptide agent is not a mini-nucleosome core protein.


SEQUENCE LISTING (198 sequences)

SEQ ID NO: 1-2 - skipped (2 entries)

SEQ ID NO: 3 - DNA, 930 bp, synthetic construct
  source 1..930: mol_type = other DNA
caggccctgtaacttttccaataaaatattctattagaaacaagttccttctagatcttcctaaaagaatactaaaggaccaaaccgtgcaacagcactggtggtattgtcttaatttagagagtccataccagtgattctcaaacattagtatgctcagagtttctggttcaataggtctgggccgggcccagggaattttcatttctaagaagttcgcaggtgatactgataactgctgttcccagggtctacattttgagatccgctgctatgtcacaaatccaaatccaaagtctttttaacaatcccttttagcccaggatgagcctaacggcaatttcacgtccttatcgccggccactttaatcttcaactgaggaggaagggagagtgagaattatgtatataatttttttcttttattttttaaaaagaaaactccaagcccacactcttaattatctgggatgaggtcatttgcacctgggtcatttatttagacaacaagtagtcttgctagaggctttaaggaggtgttagcagagcggtgtgtggcttccaggttcctgggtctaccttaaatccccatcctctttctttctttctttctttctttctttctcttttctttttcctttccgttttgaaagccccaactgcccagtctctgggtgtccgggttcccaggtccctaggtgggggcggagcccgacacggccccgccccgtctgcccgcagcgccccctccccggcccgcccgcgccggctcctccgcagtgctttcagctgtgagcttgggcggcggcggcggcggcgctccactttcggggagcccggcggctctgggaagctcactcctccactcgtacccacactcgaccgcggagcccttgcagccatgagggaacagctcaaagggtaagtgga

SEQ ID NO: 4 - DNA, 979 bp, synthetic construct
  source 1..979: mol_type = other DNA
ccggttataattaaccaggacacgtggcgaacccccctccaacacctgcccccgaacccccccatacccagcgcctcgggtctcggcctttgcggcagaggagacagcaaagcgccctctaaaaataactcctttcccggcgaccgagaccctccctgtcccccgcacagcggaaatctcccagtggcaccgagggggcgagggttaagtgggggggagggtgaccaccgcctcccacccttgccctgagtttgaatctctccaactcagccagcctcagtttcccctccactcagtccctaggaggaaggggcgcccaagcgcgggtttctggggttagactgccctccattgcaattggtccttctcccggcctctgcttcctccagctcacagggtatctgctcctcctggagccacaccttggttccccgaggtgccgctgggactcgggtaggggtgagggcccagggggcacagggggagccgagggccacaggaagggctggtggctgaaggagactcaggggccaggggacggtggcttctacgtgcttgggacgttcccagccaccgtcccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggtatggtgcgggagcagggaggtgagaggctcagctgccctccagaactcctccctggggacaacccctcccagccaatagcacagcctaggtccccctatataaggccacggctgctggcccttcctttgggtcagtgtcacctccaggatacagacagccccccttcagcccagcccagccaggtactgcacggggcgggaatctgggtgggggccagagtaggggatttctgtgggtg

SEQ ID NO: 5 - DNA, 792 bp, synthetic construct
  source 1..792: mol_type = other DNA
aagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagc

SEQ ID NO: 6-10 - skipped (5 entries)

SEQ ID NO: 11 - protein, 5 aa, synthetic construct
KRHRK

SEQ ID NO: 12 - protein, 5 aa, synthetic construct
RRRRR

SEQ ID NO: 13 - protein, 6 aa, synthetic construct
RRLARR

SEQ ID NO: 14 - protein, 11 aa, synthetic construct
KKAKAAAKPKK

SEQ ID NO: 15 - protein, 9 aa, synthetic construct
KKDGKKRKR

SEQ ID NO: 16 - protein, 5 aa, synthetic construct
KKKLK

SEQ ID NO: 17 - protein, 6 aa, synthetic construct
KKRIRK

SEQ ID NO: 18 - protein, 5 aa, synthetic construct
RKKSK

SEQ ID NO: 19 - protein, 5 aa, synthetic construct
KKPKK

SEQ ID NO: 20 - protein, 7 aa, synthetic construct
WGREERQ

SEQ ID NO: 21 - protein, 5 aa, synthetic construct
NTQIH

SEQ ID NO: 22 - protein, 7 aa, synthetic construct
WNNKTPH

SEQ ID NO: 23 - skipped

SEQ ID NO: 24 - protein, 5 aa, synthetic construct
VNRWS

SEQ ID NO: 25 - protein, 8 aa, synthetic construct
  VARIANT 1: note = X can be any amino acid
  VARIANT 5: note = X can be any amino acid
  VARIANT 6: note = X can be any amino acid
  VARIANT 8: note = X can be any amino acid
XBBBXXBX

SEQ ID NO: 26 - protein, 8 aa, synthetic construct
ARKKAAKA

SEQ ID NO: 27-28 - skipped (2 entries)

SEQ ID NO: 29 - protein, 10 aa, synthetic construct
WEPSRPFPVD

SEQ ID NO: 30 - protein, 17 aa, synthetic construct
HRRTRKAPKRIRLPHIR

SEQ ID NO: 31 - protein, 17 aa, synthetic construct
KRTGQYKLGSKTGPGQK

SEQ ID NO: 32 - protein, 4 aa, synthetic construct
KKTK

SEQ ID NO: 33 - protein, 9 aa, synthetic construct
KLRSQLVKK

SEQ ID NO: 34 - protein, 9 aa, synthetic construct
RRRCGQKKK

SEQ ID NO: 35 - skipped

SEQ ID NO: 36 - protein, 17 aa, synthetic construct
RIQNLLKITNLRIKFVK

SEQ ID NO: 37 - protein, 11 aa, synthetic construct
KKEKDIMKKTI

SEQ ID NO: 38-39 - skipped (2 entries)

SEQ ID NO: 40 - protein, 4 aa, synthetic construct
RGDS

SEQ ID NO: 41 - protein, 11 aa, synthetic construct
TTVVNPKYEGK

SEQ ID NO: 42 - protein, 11 aa, synthetic construct
ERMSQIKRLLS

SEQ ID NO: 43 - protein, 7 aa, synthetic construct
WRHRARS

SEQ ID NO: 44 - protein, 6 aa, synthetic construct
GFOGER

SEQ ID NO: 45 - protein, 5 aa, synthetic construct
LFDLM

SEQ ID NO: 46 - skipped

SEQ ID NO: 47 - protein, 7 aa, synthetic construct
QSTEKRG

SEQ ID NO: 48 - protein, 5 aa, synthetic construct
LPNTG

SEQ ID NO: 49 - skipped

SEQ ID NO: 50 - protein, 4 aa, synthetic construct
PPSY

SEQ ID NO: 51 - protein, 7 aa, synthetic construct
FEDNFVP

SEQ ID NO: 52 - protein, 4 aa, synthetic construct
YIRV

SEQ ID NO: 53 - protein, 4 aa, synthetic construct
YADW

SEQ ID NO: 54 - protein, 4 aa, synthetic construct
YTQV

SEQ ID NO: 55 - protein, 6 aa, synthetic construct
KKRPKP

SEQ ID NO: 56 - protein, 5 aa, synthetic construct
SSDDE

SEQ ID NO: 57 - protein, 5 aa, synthetic construct
RRASS

SEQ ID NO: 58 - protein, 8 aa, synthetic construct
  VARIANT 2: note = X can be any amino acid
  VARIANT 3: note = X can be any amino acid
  VARIANT 6: note = X can be any amino acid
  VARIANT 7: note = X can be any amino acid
YXXLYXXL

SEQ ID NO: 59 - protein, 5 aa, synthetic construct
LPLTG

SEQ ID NO: 60 - protein, 5 aa, synthetic construct
LAFTG

SEQ ID NO: 61 - protein, 7 aa, synthetic construct
ASSLNIA

SEQ ID NO: 62 - protein, 15 aa, synthetic construct
KKEEEKKEEEKKEEE

SEQ ID NO: 63 - protein, 7 aa, synthetic construct
LIFHKEQ

SEQ ID NO: 64 - protein, 10 aa, synthetic construct
KFNKPFVFLI

SEQ ID NO: 65 - protein, 7 aa, synthetic construct
QPEHSST

SEQ ID NO: 66 - protein, 7 aa, synthetic construct
EYHHYNK

SEQ ID NO: 67 - skipped

SEQ ID NO: 68 - protein, 6 aa, synthetic construct
GEKGEP

SEQ ID NO: 69 - protein, 5 aa, synthetic construct
KTKKK

SEQ ID NO: 70 - protein, 6 aa, synthetic construct
KALKKK

SEQ ID NO: 71 - protein, 5 aa, synthetic construct
KGKKK

SEQ ID NO: 72 - protein, 6 aa, synthetic construct
CSVTCG

SEQ ID NO: 73 - skipped

SEQ ID NO: 74 - protein, 10 aa, synthetic construct
YKYNLNGRES

SEQ ID NO: 75 - protein, 4 aa, synthetic construct
YRSL

SEQ ID NO: 76 - protein, 10 aa, synthetic construct
KGGKKKKKKK

SEQ ID NO: 77 - protein, 11 aa, synthetic construct
KKKQYTSIHHG

SEQ ID NO: 78 - protein, 4 aa, synthetic construct
KDEL

SEQ ID NO: 79 - protein, 10 aa, synthetic construct
LADQDYTKTA

SEQ ID NO: 80 - protein, 7 aa, synthetic construct
KKKYKLK

SEQ ID NO: 81 - protein, 6 aa, synthetic construct
KKRKLE

SEQ ID NO: 82 - protein, 4 aa, synthetic construct
TRSK

SEQ ID NO: 83 - protein, 6 aa, synthetic construct
HRKRKR

SEQ ID NO: 84 - protein, 6 aa, synthetic construct
NKRKRK

SEQ ID NO: 85 - protein, 7 aa, synthetic construct
AEKSKKK

SEQ ID NO: 86 - protein, 4 aa, synthetic construct
RKSK

SEQ ID NO: 87 - protein, 4 aa, synthetic construct
KRVK

SEQ ID NO: 88 - skipped

SEQ ID NO: 89 - protein, 11 aa, synthetic construct
LQQTPLHLAVI

SEQ ID NO: 90 - protein, 4 aa, synthetic construct
RRPR

SEQ ID NO: 91 - protein, 4 aa, synthetic construct
PRPR

SEQ ID NO: 92 - protein, 4 aa, synthetic construct
RPPP

SEQ ID NO: 93 - protein, 7 aa, synthetic construct
RKKRKGK

SEQ ID NO: 94 - protein, 9 aa, synthetic construct
PAAKRVKLD

SEQ ID NO: 95 - protein, 9 aa, synthetic construct
KLKIKRPVK

SEQ ID NO: 96 - protein, 7 aa, synthetic construct
PKKKRKV

SEQ ID NO: 97 - protein, 6 aa, synthetic construct
QRKRQK

SEQ ID NO: 98 - protein, 4 aa, synthetic construct
DSPE

SEQ ID NO: 99 - protein, 4 aa, synthetic construct
FQVT

SEQ ID NO: 100 - skipped

SEQ ID NO: 101 - protein, 6 aa, synthetic construct
RQGLID

SEQ ID NO: 102 - protein, 4 aa, synthetic construct
RKKH

SEQ ID NO: 103 - protein, 12 aa, synthetic construct
GRKKRRQRRRPQ

SEQ ID NO: 104 - skipped

SEQ ID NO: 105 - protein, 12 aa, synthetic construct
KSVKKRSVSEIQ

SEQ ID NO: 106 - protein, 8 aa, synthetic construct
NRRKKRAL

SEQ ID NO: 107 - protein, 5 aa, synthetic construct
KFERQ

SEQ ID NO: 108 - protein, 4 aa, synthetic construct
VRGP

SEQ ID NO: 109 - protein, 4 aa, synthetic construct
NKDS

SEQ ID NO: 110 - protein, 4 aa, synthetic construct
NRDN

SEQ ID NO: 111 - protein, 4 aa, synthetic construct
ANNR

SEQ ID NO: 112 - protein, 4 aa, synthetic construct
YTRF

SEQ ID NO: 113 - protein, 4 aa, synthetic construct
GDAY

SEQ ID NO: 114 - protein, 4 aa, synthetic construct
LLEE

SEQ ID NO: 115 - protein, 9 aa, synthetic construct
RKKRRQRRR

SEQ ID NO: 116 - protein, 4 aa, synthetic construct
YKSL

SEQ ID NO: 117 - protein, 4 aa, synthetic construct
YENF

SEQ ID NO: 118 - protein, 4 aa, synthetic construct
FQDL

SEQ ID NO: 119 - protein, 5 aa, synthetic construct
YIGSR

SEQ ID NO: 120 - protein, 5 aa, synthetic construct
IKVAV

SEQ ID NO: 121 - protein, 7 aa, synthetic construct
LIRERTE

SEQ ID NO: 122 - protein, 7 aa, synthetic construct
LVEERTQ

SEQ ID NO: 123 - protein, 6 aa, synthetic construct
IITFTK

SEQ ID NO: 124 - protein, 5 aa, synthetic construct
ILFNK

SEQ ID NO: 125 - protein, 7 aa, synthetic construct
PIRTLSK

SEQ ID NO: 126 - protein, 10 aa, synthetic construct
YGNSPLHRFK

SEQ ID NO: 127 - protein, 6 aa, synthetic construct
FFQKDR

SEQ ID NO: 128 - protein, 4 aa, synthetic construct
KSRP

SEQ ID NO: 129 - skipped

SEQ ID NO: 130 - protein, 4 aa, synthetic construct
YMKM

SEQ ID NO: 131 - protein, 6 aa, synthetic construct
RSSSFG

SEQ ID NO: 132 - protein, 8 aa, synthetic construct
LKIRGRER

SEQ ID NO: 133 - protein, 8 aa, synthetic construct
LKIRGRKR

SEQ ID NO: 134 - protein, 9 aa, synthetic construct
HVIFKKVSR

SEQ ID NO: 135 - protein, 5 aa, synthetic construct
RGPRV

SEQ ID NO: 136 - protein, 8 aa, synthetic construct
RANVKHLK

SEQ ID NO: 137 - protein, 5 aa, synthetic construct
YPKAG

SEQ ID NO: 138 - protein, 5 aa, synthetic construct
YPRTG

SEQ ID NO: 139 - protein, 4 aa, synthetic construct
DDNN

SEQ ID NO: 140 - protein, 8 aa, synthetic construct
SAVTTVVN

SEQ ID NO: 141-143 - skipped (3 entries)

SEQ ID NO: 144 - protein, 5 aa, synthetic construct
KFERQ

SEQ ID NO: 145 - protein, 41 aa, synthetic construct
KRHRKYGSSLREKRHRKLRRRRRLKRHRKKRHRKLREGGSK

SEQ ID NO: 146 - protein, 45 aa, synthetic construct
KRHRKREGSSYLREKRHRKNDLRRRRRLKRHRKKRHRKLREGGSK

SEQ ID NO: 147 - protein, 45 aa, synthetic construct
KKPKKREGSSLREYKRHRKNDLRRRRRLKRHRKKRHRKLREGGSK

SEQ ID NO: 148 - protein, 42 aa, synthetic construct
RRLARRGSSLREKRHRKYLRRRRRLKKPKKKRHRKLREGGSK

SEQ ID NO: 149 - protein, 37 aa, synthetic construct
KRHRKLREKRHRKLREKRHRKLYKRHRKKRHRKLREK

SEQ ID NO: 150 - protein, 41 aa, synthetic construct
KRHRKRILREKRHRKLREARKRHRKLKRHRKYKRHRKLREK

SEQ ID NO: 151 - protein, 36 aa, synthetic construct
KRHRKKGKKKKGEKGKKKLKGKKKLRRRRRRRQRRY

SEQ ID NO: 152 - protein, 44 aa, synthetic construct
KRHRKYAPAPKGKKKKGEKGKKKLKGKKKLKPKPRRRRRRRQRR

SEQ ID NO: 153 - protein, 42 aa, synthetic construct
KRHRKGGSGGKGKKKKGEKGKKKLKGKKKLARRRRRRRQRRY

SEQ ID NO: 154 - protein, 36 aa, synthetic construct
KRHRKYLREKRHRKRRRRRRRKRHRKYLREKRRQRR

SEQ ID NO: 155 - protein, 31 aa, synthetic construct
KRHRKKRHRKKRYVKKKRHRKRRRRRRDSLL

SEQ ID NO: 156 - protein, 36 aa, synthetic construct
KRHRKKRHRKYQKRVKKKRHRKSSSRRRRRRDSLLY

SEQ ID NO: 157 - protein, 33 aa, synthetic construct
KRHRKYKKEEEKKEEEKKEEEKRRRRRRRQRRR

SEQ ID NO: 158 - protein, 37 aa, synthetic construct
KRHRKWRKKEEEKKEEEKKEEEKYRIRRRRRRRQRRR

SEQ ID NO: 159 - protein, 30 aa, synthetic construct
KRHRKYRGDKRHRKRRRRRKRHRKTPHKKK

SEQ ID NO: 160 - protein, 34 aa, synthetic construct
KRHRKFIRGDYKRHRKRRRRRKRHRKLATPHKKK

SEQ ID NO: 161 - protein, 37 aa, synthetic construct
KRHRKRGDKRHRKRRRRRKRHRKGSSRNTPHYQKKKK

SEQ ID NO: 162 - protein, 34 aa, synthetic construct
KRHRKRGDKRHRKLKRHRKRRRRKRHRKYTPHKK

SEQ ID NO: 163 - protein, 31 aa, synthetic construct
KRHRKRGDKRHRKKRHRKKRHRKYRGDKKTK

SEQ ID NO: 164 - protein, 33 aa, synthetic construct
KRHRKRGDKKRKKKKRGDYKKRRRRRKKKPPSY

SEQ ID NO: 165 - protein, 33 aa, synthetic construct
KRHRKRKRKRKRRRRRKKKRYASSLNIAKRRRR

SEQ ID NO: 166 - protein, 33 aa, synthetic construct
KRKKRKGKRLKRRREKRHRKRASSLNIAYKKKK

SEQ ID NO: 167 - protein, 33 aa, synthetic construct
KRKKRRLKRKRKRRRRREKRHRKRRRQRRRKKY

SEQ ID NO: 168 - protein, 32 aa, synthetic construct
KRKKRRYKRKRRRRRKRHRKLRERKRRLREKK

SEQ ID NO: 169 - protein, 36 aa, synthetic construct
KRKYNGRKRKRKKRHRKKKKRRRRKRHRKYNGRKKK

SEQ ID NO: 170 - protein, 30 aa, synthetic construct
KRHRKYWRHRARSKRHRKKKKKKRKKRKGK

SEQ ID NO: 171 - protein, 32 aa, synthetic construct
KRHRKRGDKRHRKKKKYNRRKKRALRKKRKGK

SEQ ID NO: 172 - protein, 39 aa, synthetic construct
KKRKRGGKTKKKAKKALKKKKKGKKKKRRRRKYKAAPKK

SEQ ID NO: 173 - protein, 43 aa, synthetic construct
KKKAYPKALKKPKKKKKAYPKALKRRRRRKNRRKKRALKRHRK

SEQ ID NO: 174 - protein, 40 aa, synthetic construct
KTRSKKKKKRGDKKKKNRRKKRALYNTQIHKKKKKAAPKK

SEQ ID NO: 175 - protein, 35 aa, synthetic construct
KGKKKKGEKGKKKLKGKKKLRRRRRYSPKKRRQRR

SEQ ID NO: 176 - protein, 36 aa, synthetic construct
KRHRKYLREKRHRKLRRRRRLKRHRKKRHRKYLREK

SEQ ID NO: 177 - protein, 39 aa, synthetic construct
KRHRKYLREKRHRKYLREKRHRKLKRHRKKRHRKLYREK

SEQ ID NO: 178 - protein, 36 aa, synthetic construct
KRHRKYKGKKKKGEKGKKKLKGKKKLRRRRRRRQRR

SEQ ID NO: 179 - protein, 35 aa, synthetic construct
KRHRKLREKRHRKRRRRRRRKRHRKLREKRRQRRY

SEQ ID NO: 180 - protein, 31 aa, synthetic construct
KRHRKKRHRKKRVKKKRHRKRRRRRRDSYLL

SEQ ID NO: 181 - protein, 33 aa, synthetic construct
KRHRKKKEEEKKEEEKKEEEKYRRRRRRRQRRR

SEQ ID NO: 182 - protein, 37 aa, synthetic construct
KRHRKYQSKKEEEKKEEEKKEEEKNQRRRRRRRQRRR

SEQ ID NO: 183 - protein, 31 aa, synthetic construct
KRHRKYRGDKRHRKRRRRRKRHRKYTPHKKK

SEQ ID NO: 184 - protein, 34 aa, synthetic construct
KRHRKRGDKRHRKLKRHRKRRRRKRHRKTPHKKY

SEQ ID NO: 185 - protein, 31 aa, synthetic construct
KRHRKRGDKRHRKYKRHRKKRHRKRGDKKTK

SEQ ID NO: 186 - protein, 34 aa, synthetic construct
KRHRKYRGDKKRKKKKYRGDKKRRRRRKKKPPSY

SEQ ID NO: 187 - protein, 39 aa, synthetic construct
KRHRKYGGSRGDKKRKKKKRGDSSSKKRRRRRKKKPPSY

SEQ ID NO: 188 - protein, 33 aa, synthetic construct
KRHRKRKRKRKRRRRRKKKRASSLNIAKRRRRY

SEQ ID NO: 189 - protein, 33 aa, synthetic construct
KRKKRKGKRLKRRREYKRHRKRASSLNIAKKKK

SEQ ID NO: 190 - protein, 33 aa, synthetic construct
KRKKRRLYKRKRKRRRRREKRHRKRRRQRRRKK

SEQ ID NO: 191 - protein, 32 aa, synthetic construct
KRKKRRKRKRRRRRKRHRKLRERKRRLREKKY

SEQ ID NO: 192 - protein, 35 aa, synthetic construct
YKRKNGRKRKRKKRHRKKKKRRRRKRHRKNGRKKK

SEQ ID NO: 193 - protein, 41 aa, synthetic construct
KRKYWRNGRKRKRQKRHRKKKKRARRRRKRHRKNGRKHKKK

SEQ ID NO: 194 - protein, 30 aa, synthetic construct
KRHRKYWRHRARSKRHRKKKPKKRKKRKGK

SEQ ID NO: 195 - protein, 37 aa, synthetic construct
KRHRKPKPRIWRHRARSRDYKRHRKKKPKKRKKRKGK

SEQ ID NO: 196 - protein, 32 aa, synthetic construct
  MOD_RES 28: note = Sulfated tyrosine
KARHRKGGSLIRGDKELKRPPRRRRKHYNKSR

SEQ ID NO: 197 - protein, 32 aa, synthetic construct; identical to SEQ ID NO: 196

SEQ ID NO: 198 - protein, 35 aa, synthetic construct
YKRHRKLREKRHRKLRRRRRLKRHRKKRHRKLREK